(19) United States
(12) Patent Application Publication
(10) Pub. No.: US 2023/0314412 A1
(43) Pub. Date: Oct. 5, 2023

(54) Neuronal diencephalon stem cells, preparation and uses for treatment and prevention of hormonal disorders and other diseases

(71) Applicant: Florica Therapeutics, Inc., San Francisco, CA (US)

(72) Inventor: Christine Ichim, Livermore, CA (US)

(21) Appl. No.: 17/913,403

(22) PCT Filed: Mar. 19, 2021
(86) PCT No.: PCT/US2021/023308

§ 371 Date: Sep. 21, 2022

Related U.S. Application Data
(60) Provisional application No. 62/992,907, filed on Mar. 21, 2020.
(60) Provisional application No. 62/994,838, filed on Mar. 25, 2020.
(60) Provisional application No. 62/994,843, filed on Mar. 26, 2020.
(60) Provisional application No. 62/994,863, filed on Mar. 26, 2020.

(51) Int. Cl. G01N 33/50

(52) U.S. Cl. CPC G01N 33/5058

(57) ABSTRACT

Methods of making neuronal stem cells and exosomes of diencephalon lineage are disclosed. Also provided are compositions comprising neuronal stem cells or exosomes of diencephalon lineage, which may be formulated as pharmaceutical formulations for the treatment and prevention of disorders associated with the neuroendocrine system and the control of behavioral and physiological processes.

### CROSS-REFERENCE TO RELATED APPLICATIONS

The present application claims priority to U.S. Provisional Application No. 62/994,863, filed Mar. 26, 2020, U.S. Provisional Application No. 62/994,838, filed Mar. 25, 2020, U.S. Provisional Application No. 62/994,843, filed Mar. 26, 2020, and U.S. Provisional Application No. 62/992,907, filed Mar. 21, 2020, the contents of which are incorporated by reference in their entirety.

### INCORPORATION OF SEQUENCE LISTING

A computer readable form of the Sequence Listing, “9214-063649_Sequence.txt” (3,596 bytes) created on Jun. 22, 2023, is herein incorporated by reference. The Sequence Listing contains no new matter. A copy of the “Sequence Listing” is available in electronic form from the USPTO website. An electronic copy of the “Sequence Listing” will also be available from the USPTO upon request and payment of the fee set for in 37 CFR 1.19(b)(3).

### FIELD OF INVENTION

Methods of making neuronal stem cells and exosomes of diencephalon lineage are disclosed. Also provided are compositions comprising neuronal stem cells or exosomes of diencephalon lineage, which may be formulated as pharmaceutical formulations for the treatment and prevention of disorders associated with the neuroendocrine system and the control of behavioral and physiological processes.

### BACKGROUND OF INVENTION

The hypothalamus is a small region of the brain, located at the base of the brain, near the pituitary gland, and known to play a crucial role in various functions, including hormone release, body temperature regulation, daily physiological cycle maintenance, appetite control, sexual behavior and emotional responses.

The anterior region of the hypothalamus, the supraoptic region, is involved in the secretion of hormones, such as corticotropin-releasing hormone (CRH), thyrotropin-releasing hormone (TRH), gonadotropin-releasing hormone (GnRH), oxytocin, vasopressin and somatostatin, and in maintaining circadian rhythms. The middle region of the hypothalamus, the tuberal region, regulates appetite and the growth hormone-releasing hormone (GHRH). The posterior area of the hypothalamus, the mammillary region, regulates body temperature.

Because of their strategic location, hypothalamic neurons can sense both neural and physiological signals, and respond by releasing neurotransmitters and neuromodulators into the brain. Human hypothalamic neuron dysfunction has been related to a variety of diseases, such as obesity, hypertension, and mood and sleep disorders. Therefore, the production of human hypothalamic neurons in vitro can be very helpful in the understanding and treatment of hypothalamic neuron dysfunction-related diseases in humans.

However, the current technologies for creating, manufacturing and scaling up the production of human hypothalamic stem cells and exosomes thereof suffer from several drawbacks.

Hypothalamic extracellular vesicles and exosomes are produced from stem progenitor and mature cells. The production of extracellular vesicles or exosomes from human hypothalamus cells requires a human hypothalamic cell source. However, hypothalamic cells are difficult to obtain, and it is exceedingly difficult to source human hypothalamic stem cells. This problem is due, in part, to the fact that the hypothalamus constitutes only 0.3% of the brain and obtaining human hypothalamic cell sources requires access to cadavers. Obtaining human hypothalamic stem cells is even more challenging, as the process requires the use of a child cadaver.

Methods aimed at devising culture conditions that stimulate the differentiation of pluripotent cells into neurons of hypothalamus lineage are not reproducible across pluripotent cell lines and give rise to mature hypothalamus cells of low purity. It is not currently known whether human hypothalamus stem cells exist, and what culture conditions are necessary for their production. The identification of the specific stage at which hypothalamus stem cells are formed along this differentiation pathway is particularly challenging. Furthermore, commercial large-scale manufacture of these cells has not yet been developed, and the conditions that allow for the isolation of extracellular vesicles or exosomes from human hypothalamic stem and progenitor cells derived from pluripotent cells have not been defined.

Alternative economically viable solutions for the creation, manufacturing and mass production of human hypothalamic stem and/or progenitor cells and extracellular vesicles or exosomes of such cells are therefore needed.

### SUMMARY OF THE INVENTION

The present application presents a solution to the aforementioned challenges by providing reliable, cost-effective and easily scalable methods for isolating and purifying human hypothalamus stem cells and exosomes during induced pluripotent cell in vitro differentiation. The human hypothalamus stem cells and exosomes obtained by the disclosed methods may be formulated as pharmaceutical formulations or as loaded vectors for targeted delivery of drugs or active compounds for the treatment and prevention of disorders associated with the neuroendocrine system and the control of behavioral and physiological processes.

Thus, in some embodiments, provided herein is a method for identifying, isolating and purifying human hypothalamus stem cells at an intermediate transitory stage during in vitro induced pluripotent stem cell differentiation, which corresponds to human hypothalamus stem cell formation. The disclosed method comprises: (i) culturing induced pluripotent stem cell in culture media and enabling reproducible differentiation of induced pluripotent stem cells into cells of ventral diencephalon hypothalamic cell lineage; (ii) identifying hypothalamic cells between day O and day 15 of cell culture that display hypothalamus markers and neuronal stem cell markers; (iii) isolating hypothalamus stem cells at hypothalamus marker and neuronal stem cell marker maximal expression; and (iv) purifying isolated human hypothalamus stem cells.

Neuronal stem cell markers that may be used to detect human hypothalamus stem cell formation include, but are not limited to, one or more of Sox2+, Bmi-1+, nestin+, Musashil+Cxcr4+.

Hypothalamus markers that may be used to detect human hypothalamus stem cell formation include, but are not limited to, one or more of NK2 homeobox 1 (Nkx2. l) and homeobox protein orthopedia.

In some embodiments, the purified human hypothalamus stem cells obtained by the disclosed method are Raxhi, Soxl¹⁰, Sox2+ and Bmi-1+_

In some embodiments, hypothalamus marker and neuronal stem cell marker maximal expression is between day 7 and day 15 of cell culture.

In some embodiments, the purified human hypothalamus stem cells obtained by the disclosed method are tanycytes.

In some embodiments, the disclosed method may further comprise analyzing the purified human hypothalamus stem cells for immunophenotype, neurosphere formation and ability to give rise to mature hypothalamus neurons.

In some embodiments, the ability to give rise to mature hypothalamus neurons is measured by detecting expression of one or more neuropeptide markers. Suitable neuropeptide markers include, but are not limited to, Otp, Rax, neuropeptide Y (NPY), cocaine amphetamine regulated transcript (CART), a-melanocyte stimulating hormone (a-MSH), neuropeptide Y receptor Y2 (NPYR), ghrelin receptor (GhrR), and melanin concentrating hormone (MCH).

In some embodiments, the disclosed method may further comprise isolating and purifying exosomes from the culture media, and analyzing the purified exosomes. Suitable techniques for analyzing the purified exosomes include, but are not limited to, western blot analysis, flow cytometry, nanoparticle-tracking analysis, density-gradient ultracentrifugation, pull-down assay, and real-time PCR.

In other embodiments, provided herein are purified human hypothalamus exosomes obtained by the disclosed method.

In some embodiments, the disclosed method may further comprise engineering the purified exosomes to express one or more foreign molecules. Suitable foreign molecules include, but are not limited to, growth factors, nucleic acids, cytokines, vaccines, inactivated viral proteins, drugs, chemotherapeutics and biologically active molecules.

In some embodiments, the nucleic acid is DNA, messenger RNA, micro RNA, or small interfering RNA

In some embodiments, the disclosed method may further comprise administering to a subject in need thereof a pharmaceutical composition comprising the engineered exosomes produced according to the disclosed method, for preventing, treating or controlling a disease or disorder in the subject.

In additional embodiments, provided herein is a method for identifying a drug that modulates hypothalamus function in a subject in need thereof. The disclosed method comprises (a) transfecting purified human hypothalamus stem cells with a reporter gene operably linked to a transcriptional regulatory element isolated from the subject and regulating expression of the reporter gene; (b) contacting transfected human hypothalamus stem cells with a candidate drug; (c) determining drug effect on transcriptional regulatory element expression by detecting and measuring a signal emanating from the reporter gene and comparing the signal to a signal emanated by the reporter gene prior to contact with the drug, thereby identifying a drug that modulates hypothalamus function in the subject.

In some embodiments, the effect of the drug may be further determined by measuring one or more properties, such as cell viability, proliferation, apoptosis, stern-ness, and differentiation.

In some embodiments, the candidate drug may decrease neuroinflammation, alter circadian rhythm, regulate thyroid function or affect obesity, inflammation, infertility or aging.

In some embodiments, the subject is a mammal. In some embodiments, the subject is a human subject.

The methods provided herein present several attractive features and desirable properties that make them suitable for use. For example, the disclosed method allows detection and purification of human hypothalamus stem cells at a precise stage during pluripotent or embryonic stem cell differentiation, when expression of hypothalamus markers and neuronal stem cell markers reaches its pick. The disclosed method is therefore precise, reliable and quick, as it reproducibly detects and isolates hypothalamus stem cells, and it requires less purification and validation steps. Furthermore, the disclosed method is easily scalable for large-scale production of human hypothalamus stem cells and exosomes. In addition, because disorders associated with the neuroendocrine system and the control of behavioral and physiological processes can be targeted by the disclosed human hypothalamus stem cell and exosome compositions, optimal treatment plans can be effectively devised and developed.

The foregoing and other features of the disclosure will become more apparent from the following detailed description of several embodiments, which proceeds with reference to the accompanying figures.

### BRIEF DESCRIPTION OF THE FIGURES

FIG. 1 shows a hypothalamus stem and progenitor cell isolation process scheme as provided herein. Pluripotent cells are cultured in media until they develop neuronal and hypothalamic differentiation factors. These factors allow the identification of cells with stem cell properties and the production of mature arcuate hypothalamus neurons.

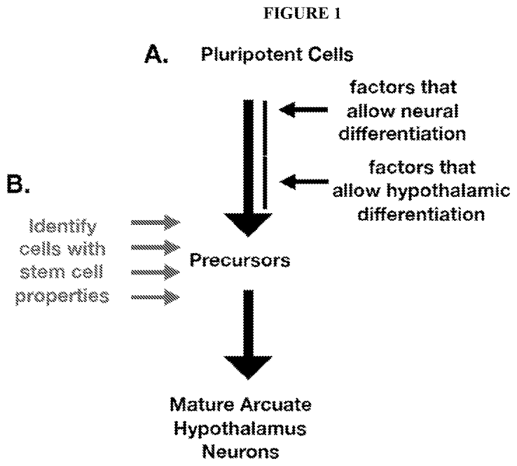

FIG. 2 shows the morphology of the induced pluripotent stem cell line WTCl 1, which can be differentiated into hypothalamus stem and progenitor cells by the process provided herein.

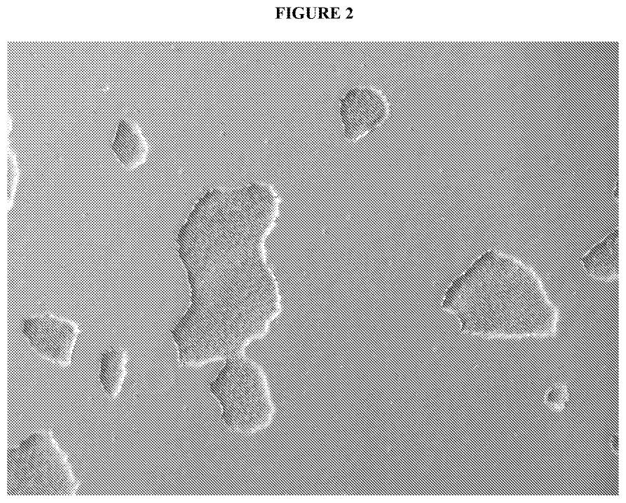

FIG. 3 shows the morphology of the induced pluripotent stem cell line WTCl 1, which can be differentiated into hypothalamus stem and progenitor cells by the process provided herein.

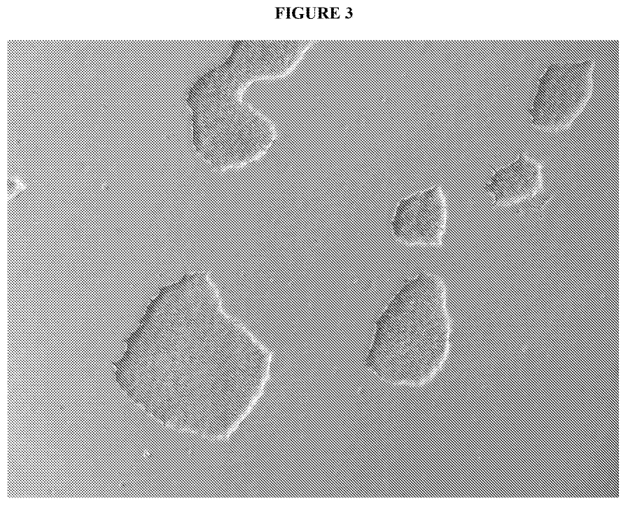

FIG. 4 shows the morphology of the induced pluripotent stem cell line WTCl 1, which can be differentiated into hypothalamus stem and progenitor cells by the process provided herein.

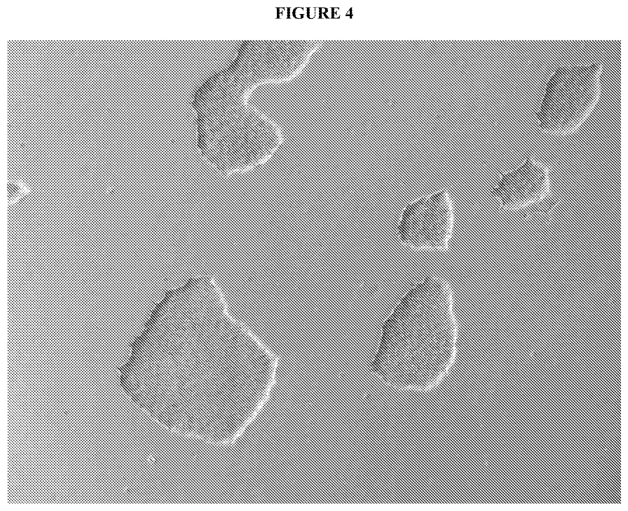

FIG. 5 shows the morphology of the induced pluripotent stem cell line WTCl 1, which can be differentiated into hypothalamus stem and progenitor cells by the process provided herein.

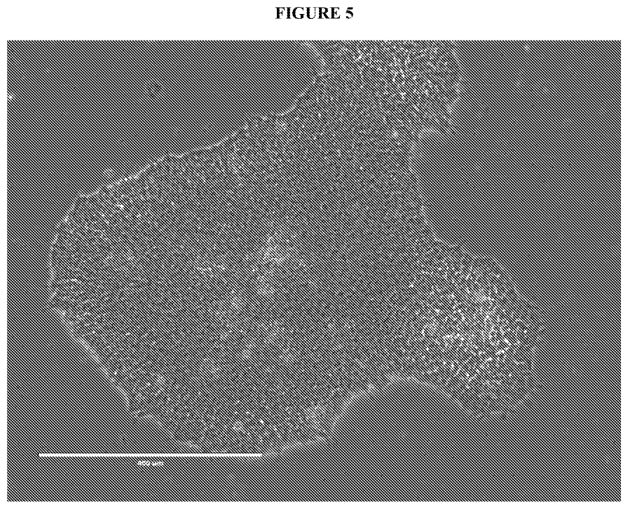

FIG. 6 shows a hypothalamus stem and progenitor cell isolation process scheme as provided herein. Embryonic stem cells or pluripotent cells are induced to differentiate in culture media under conditions that stimulate cell differentiation toward a hypothalamus cell lineage, such as Sonic HedgeHog activation (SHH) signaling pathway and SMAD and NOTCH signaling pathway inhibition, until the cells begin to express neuronal and hypothalamic differentiation markers. Cells that express markers of both neuronal stem/progenitor cells and of the hypothalamus cell lineage are identified, collected and used to produce mature arcuate hypothalamus neurons, and extracellular vesicles and exosomes thereof.

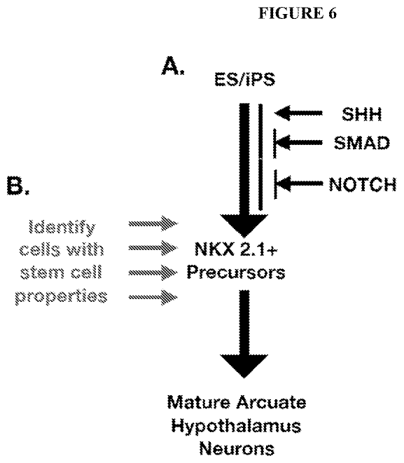

FIG. 7: (A) Human hypothalamus stem cells created by the disclosed method contain markers of both neural stem cells and cells of the hypothalamus lineage, as shown by immunofluorescence. (B) In contrast, undifferentiated parental pluripotent stem cells do not contain markers of neural stem cells or cells of the hypothalamus lineage, as shown by the lack of immunofluorescence.

FIG. 8: (A) Human hypothalamus stem cells created by the disclosed method contain markers of both neural stem cells and cells of the hypothalamus lineage, as shown by immunofluorescence. (B) In contrast, undifferentiated parental pluripotent stem cells do not contain markers of neural stem cells or cells of the hypothalamus lineage, as shown by the lack of immunofluorescence.

FIG. 9: Human hypothalamus stem cell exosomes are labeled using fluorescent dyes and detected. (A) Fluorescently labeled human hypothalamus stem cell exosomes are quantified using fluorescent nanoparticle tracking analysis (fNTA). (B) Liposomes containing 0.1 μM fluorescent polystyrene beads are used as control.

FIG. 10 shows RNA contained in human hypothalamus stem cell exosomes is stained with SYTO RNASelect stain.

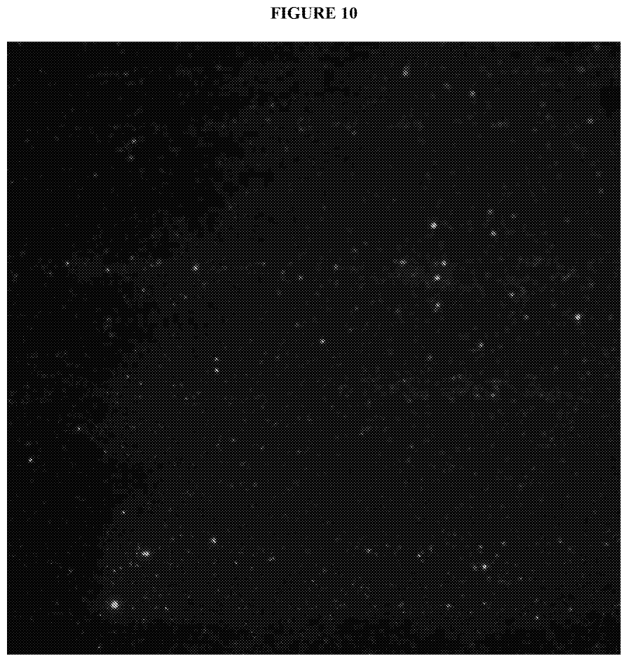

FIG. 11 shows the membrane of human hypothalamus stem cell exosomes stained with BODIPY TR ceramide.

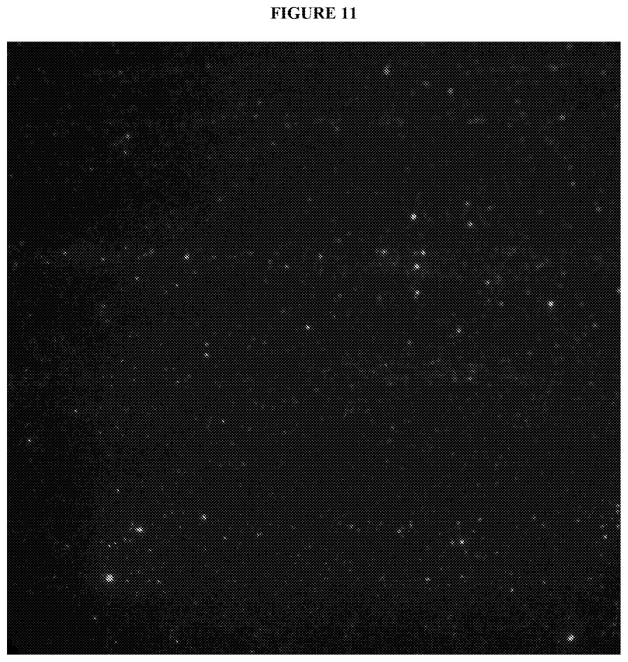

FIG. 12 shows the staining of both RNA and exosomal membrane in exosomal particles isolated from human hypothalamus stem cells. The double staining of both RNA and exosomal membrane provides evidence that the vesicles isolated from human hypothalamus stem cells are in fact exosomes, and not artifacts.

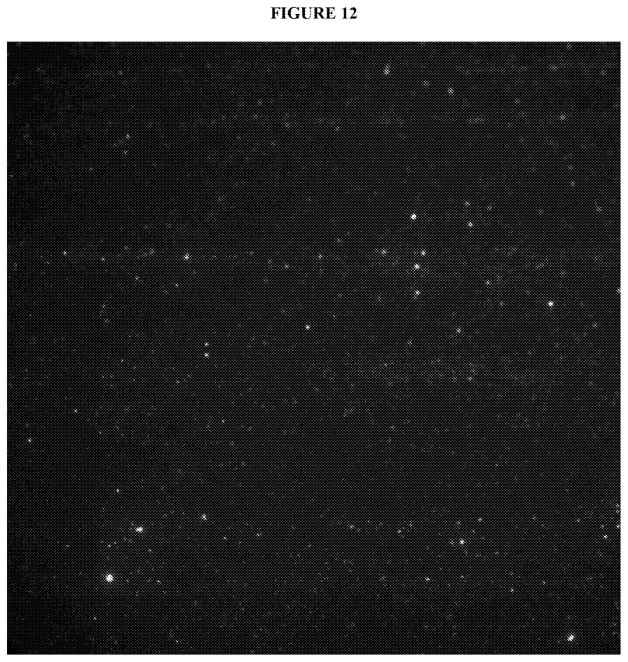

FIG. 13 shows transmission electron microscopy (TEM) images of human hypothalamus stem cells produced by the disclosed method.

FIG. 14 shows transmission electron microscopy (TEM) images of human hypothalamus stem cells produced by the disclosed method.

FIG. 15 shows transmission electron microscopy (TEM) images of human hypothalamus stem cells produced by the disclosed method.

FIG. 16 shows transmission electron microscopy (TEM) images of human hypothalamus stem cells produced by the disclosed method.

FIG. 17: capillary western blotting images of human hypothalamus stem cells produced by the disclosed method show that exosomes produced from human hypothalamus stem cells contain proteins associated with the exosomes.

FIG. 18 shows that human hypothalamus stem cell exosomes produced by the disclosed method reduce the expression of inflammatory genes in human cells.

FIG. 19 shows that human hypothalamus stem cells loaded with the thyroid hormone T3 activate the thyroid hormone receptor, as measured by qPCR of the thyroid hormone receptor target gene ectonucleotide pyrophosphatase/phosphodiesterase 2 (ENPP2).

FIG. 20 shows fluorescent staining of exogenous siRNA loaded into human hypothalamus stem cells, indicating that human hypothalamus stem cells were successfully engineered to express exogenous siRNA molecules.

FIG. 21 shows that human hypothalamus stem cells engineered to express IL-6 siRNA silence the expression of IL-6 in human cells.

FIG. 22 shows that human hypothalamus stem cell exosomes loaded with various NFKB inhibitors are able to cross an in vitro model of a blood brain barrier and suppress activation of NFKB. Negative control: vehicle control: HSC Exosome: human hypothalamus stem cell exosomes; Loaded exosome A: human hypothalamus stem cell exosomes loaded with NFKB inhibitor 11-CAS 749886-87-1; Loaded exosome B: human hypothalamus stem cell exosomes loaded with NFKB inhibitor 111-CAS 380623-76-7; Loaded exosome C: human hypothalamus stem cell exosomes loaded with NFKB inhibitor IV-CAS 139141-12-1.

### DETAILED DESCRIPTION

The following explanations of terms and methods are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. For example, reference to “comprising a therapeutic agent” includes one or a plurality of such therapeutic agents. The term “or” refers to a single element of stated alternative elements or a combination of two or more elements, unless the context clearly indicates otherwise. For example, the phrase “A or B” refers to A, B, or a combination of both A and B.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In some examples, the numbers expressing quantities of ingredients, properties such as molecular weight, reaction conditions, and so forth, used to describe and claim certain embodiments are to be understood as being modified in some instances by the term “about” or “approximately.” Notwithstanding that the numerical ranges and parameters setting forth the broad scope of some examples are approximations, the numerical values set forth in the specific examples are reported as precisely as practicable. The recitation of ranges of values herein is merely intended to serve as a shorthand method of referring individually to each separate value falling within the range.

To facilitate review of the various embodiments of this disclosure, the following explanations of specific terms are provided:

Administer: To provide or give a subject a composition by an effective route. Application is local. Exemplary routes of application include, but are not limited to, oral and topical routes.

Adult Stem Cell or Somatic Stem Cell: An undifferentiated cell found in a differentiated tissue and capable of renewing itself and under specific conditions differentiate into a specialized cell type of the tissue from which it originated. Adult stem cells are multipotent. Non-limiting examples of adult stem cells include hematopoietic stem cells, bone marrow-derived stem cells, neural stem cells (NSC), and multipotent stem cells derived from epithelial and adipose tissues and umbilical cord blood (UCB).

Antibiotic: A chemical substance capable of treating bacterial infections by inhibiting the growth of, or by destroying existing colonies of bacteria and other microorganisms.

Anti-inflammatory agent: An active agent that reduces inflammation and swelling.

Anti-Oxidant: An active agent that inhibits oxidation or reactions promoted by oxygen or peroxides.

Cell Differentiation: A process by which a less specialized cell, such as a stem cell, develops or matures or differentiates into a more specialized cell or a differentiated cell.

Contacting: Placement in direct physical association; includes both in solid and liquid form.

Control: A reference standard. In some examples, a control is a known value or range of values, such as one indicative of the presence or the absence of a disease. In some examples, a control is a value or range of values, indicating a response in the absence of a therapeutic agent.

Effective amount: The amount of an active agent (alone or with one or more other active agents) sufficient to induce a desired response, such as to prevent, treat, reduce and/or ameliorate a condition. Effective amounts of an active agent, alone or with one or more other active agents, can be determined in many different ways, such as assaying for a reduction in of one or more signs or symptoms associated with the condition in the subject or measuring the level of one or more molecules associated with the condition to be treated.

Embryonic Stem Cell: A cell derived from an embryo at the blastocyst stage or before substantial differentiation of the cell into the three germ layers that displays morphological characteristics of undifferentiated cells, and that is capable of self-renewing. Exemplary morphological characteristics of undifferentiated cells include, but are not limited to, high nuclear/cytoplasmic ratios and the presence of prominent nucleoli under a microscope. Under appropriate conditions, embryonic stem cells can differentiate into cells or tissues that are derivatives of each of the three germ layers: endoderm, mesoderm, and ectoderm. Assays for the identification of an embryonic stem cell include, but are not limited to, the ability to form teratoma in a suitable host and to be stained for markers of an undifferentiated cell, such as Oct-4.

Exosome or Extracellular Vesicle (EV): An extracellular vesicle that contains constituents of the cell that secretes it. Exosomes have a size from about 10 nm to about 10 μm and consist of fluids, macromolecules, solutes, and metabolites derived from the cells that secrete them. They are contained within a lipid bilayer or micelle. Exosomes or extracellular vesicles may also include lipid vesicle engineered to contain bioactive molecules, such as a neurons, as well as ectosomes. Exosomes are released on the exocytosis of multivesicular bodies (MVBs). Ectosomes are vesicles assembled at and released from the plasma membrane. The size of the extracellular vesicles may be in a range from about 20 nm to about 10 m, or from about 20 nm to about 1 μm, or from about 20 nm to about 500 nm, or from about 30 nm to about 100 nm, or from about 30 nm to about 160 nm, or from about 80 nm to about 160 nm. In some embodiments, the EVs are exosomes that are from about 20 nm to about 150 nm in size.

Induced Pluripotent Stem Cells (iPS or iPSCs): Pluripotent stem cells artificially generated from a non-pluripotent cell, typically an adult somatic cell, or from a terminally differentiated cell, such as a fibroblast, a hematopoietic cell, a myocyte, a neuron, an epidermal cell, or the like. Human induced pluripotent stem cells are produced from somatic cells, such as dermal fibroblasts, taken from a human individual. Pluripotent stem cells typically display the characteristic morphology of human embryonic stem cells (hESCs), express the pluripotency-associated markers SSEA-4 and TRAl-60, the transcription factors Oct-4 and Nanog, and differentiate in vitro into cell types derived from each of the three embryonic germ layers. Pluripotent stem cells may be an established cell line produced from somatic cells taken from a subject, or derived from any human somatic cell. Suitable somatic cells include, but are not limited to, keratinocytes, dermal fibroblasts and leukocytes derived from peripheral blood.

Inhibiting a condition: Reducing, slowing, or even stopping the development of a condition, for example, in a subject who is at risk of developing or has a particular condition.

Localized application: The application of an active agent in a particular location in the body.

Mucosal Administration: Administration through the mouth, nose, vagina, eyes and ears of a subject.

Oral administration: Delivery of an active agent through the mouth.

pH Adjuster or Modifier: A molecule or buffer used to achieve desired pH control in a formulation. Exemplary pH modifiers include acids (e.g., acetic acid, adipic acid, carbonic acid, citric acid, fumaric acid, phosphoric acid, sorbic acid, succinic acid, tartaric acid, basic pH modifiers (e.g., magnesium oxide, tribasic potassium phosphate), and pharmaceutically acceptable salts thereof.

Pharmaceutically acceptable carriers: The pharmaceutically acceptable carriers useful in this disclosure are conventional. The nature of the carrier can depend on the particular mode of administration being employed. For instance, oral applications usually include pharmaceutically and physiologically acceptable fluids such as water, physiological saline, balanced salt solutions, aqueous dextrose, glycerol or the like as a vehicle. In addition to biologically-neutral carriers, oral compositions may also contain minor amounts of non-toxic auxiliary substances, such as wetting or emulsifying agents, preservatives, and pH buffering agents and the like.

Stem Cell: A cell having the potential to self-renew and indefinitely divide, and, under appropriate conditions, differentiate into a dedicated progenitor cell or a specified cell or tissue. Stem cells can be pluripotent or multipotent. Stem cells include, but are not limited to embryonic stem cells, embryonic germ cells, cloned stem cells from somatic nuclei, adult stem cells, and umbilical cord blood cells. The term “stem cells” encompasses mammalian stem cells, including human stem cells, and non-human mammalian stem cells, including, but not limited to, those derived from primates, ungulates, ruminants and rodents. When cultured under suitable conditions and/or contacted with, or exposed to, particular compounds and/or conditions, stem cells may differentiate into any one of the specialized cell types which form embryonic and/or adult tissues.

Subject: A living multi-cellular vertebrate organism, a category that includes human and non-human mammals, as well as birds (such as chickens and turkeys), fish, and reptiles. Exemplary subjects include mammals, such as human and non-human primates, rats, mice, dogs, cats, rabbits, cows, pigs, goats, horses, and the like.

Surface or Body Surface: A surface located on the human body or within a body orifice. Thus, a “body surface” includes, by way of example, skin, teeth, skin or mucosal tissue, including the interior surface of body cavities that have a mucosal lining.

Tanycytes: Cells in the hypothalamus that have stem cell properties.

Topical administration: Delivery of an active agent to a body surface, such as, the skin or mucosa, as in, for example, topical drug administration in the prevention or treatment of various skin disorders.

Under conditions sufficient to: A phrase that Is used to describe any environment that permits the desired activity.

### Method for Producing Human Hypothalamus Stem Cells and Exosomes from Induced Pluripotent or Embryonic Stem Cells

The hypothalamus is a small, but essential region of the brain. Hypothalamic neurons respond to physiological stimuli by releasing neurotransmitters and neuromodulators into the brain, and regulate several physiological and behavioral functions, including immune and hormonal function, reproduction, stress, circadian rhythms, sleep pattern, mood status and social behavior.

Hypothalamus dysfunction has been linked to metabolic and inflammatory diseases, such as obesity and neuro-inflammation, and to sleep and mood disorders. In vitro generation of hypothalamic stem cells and neurons is vital for the study and understanding of these diseases and disorders, and for the development of appropriate therapies. The unavailability of human hypothalamic stem cells and inconsistently reproducible protocols are major obstacles to progressive research in this field.

Provided herein is an approach that overcomes these challenges, by providing a reliable, cost-effective and easily scalable method that allows isolation and purification of human hypothalamus stem cells and exosomes at a precise intermediate stage during induced pluripotent or embryonic stem cell in vitro differentiation, just as the developing hypothalamus stem cells reach maximal expression of hypothalamus markers and neuronal stem cell markers maximal expression. The disclosed method is highly efficient, as hypothalamic stem cells can be grown, isolated and purified in a short time, between day 7 and day 15 of cell culture. The hypothalamic stem cells produced in vitro by the method provided herein are morphologically similar to hypothalamic stem cells produced in vivo, including tanycytes, which are neural stem cells or progenitor cells found in the postnatal hypothalamus. The hypothalamic stem cells produced by the disclosed method can be used for the production of exosomes and the development of drug screens and therapies for hypothalamus-related diseases and disorders.

Thus, provided herein is a method for identifying, isolating and purifying human hypothalamus stem cells at an intermediate transitory stage during in vitro induced pluripotent stem cell differentiation, which corresponds to human hypothalamus stem cell formation. The disclosed method comprises: (i) culturing induced pluripotent stem cell in culture media and enabling reproducible differentiation of induced pluripotent stem cells into cells of ventral diencephalon hypothalamic cell lineage; (ii) identifying hypothalamic cells between day O and day 15 of cell culture that display hypothalamus markers and neuronal stem cell markers; (iii) isolating hypothalamus stem cells at hypothalamus marker and neuronal stem cell marker maximal expression; and (iv) purifying isolated human hypothalamus stem cells.

To stimulate differentiation toward hypothalamic lineage and decrease cell variability, the pluripotent or embryonic stem cells are cultured in presence of small molecules and peptide modulators that block the BMP and the TGF-pathways, and the canonical Wnt pathways.

Human hypothalamus stem cell formation is identified by detecting the expression of neuronal stem cell markers, such as Sox2+ and Bmi-1+, hypothalamus markers, such as NK2 homeobox 1 (Nkx2. l) and homeobox protein orthopedia; Raxhi and Sox1¹⁰; and proteins that are mainly expressed in nerve cells, such as neuroectodermal stem cell marker ((nestin)+, Musashi RNA binding protein 1 (Musashilt and C-X-C motif chemochine receptor 4 (Cxcr4t.

The disclosed method may further comprise analyzing the purified human hypothalamus stem cells for immunophenotype, neurosphere formation and ability to give rise to mature hypothalamus neurons.

In some embodiments, the ability to give rise to mature hypothalamus neurons is measured by detecting expression of one or more neuropeptide markers. Suitable neuropeptide markers include, but are not limited to, Otp, Rax, neuropeptide Y (NPY), cocaine amphetamine regulated transcript (CART), a-melanocyte stimulating hormone (a-MSH), neuropeptide Y receptor Y2 (NPYR), ghrelin receptor (GhrR), and melanin concentrating hormone (MCH).

Additionally provided herein is the production of exosomes from hypothalamic stem cells. Thus, in some embodiments, the disclosed method may further comprise isolating and purifying exosomes from the stem cell culture media, and analyzing the purified exosomes by one or more suitable techniques, such as, for example, western blot analysis, flow cytometry, nanoparticle-tracking analysis, density-gradient ultracentrifugation, pull-down assay, and real-time PCR.

The purified human hypothalamus exosomes thus obtained may be engineered to express one or more foreign molecules. Suitable foreign molecules include, but are not limited to, growth factors, nucleic acids, cytokines, vaccines, inactivated viral proteins, drugs, chemotherapeutics and biologically active molecules.

Suitable nucleic acids include, but are not limited to, DNA, messenger RNA, micro RNA, or small interfering RNA (siRNA), such as siRNA molecules targeting one or more genes, such as toll-like receptor 4 (TLR-4), NFkappaB, and tumor necrosis factor alpha; interleukins, such as interleukin-I and interleukin-6; proteins, such as fatty acid binding protein Fh 15; and interferons, such as IFN-alpha and IFN-gamma.

Suitable growth factors include, but are not limited to, growth factors involved in organism development, angiogenesis and wound healing, such as human growth hormone (hGH), platelet-derived growth factor-BB (PDGF-BB), and bone morphogenetic protein (BMP).

Suitable cytokines include, but are not limited to, interleukins, interferons, and synthetic cytokines.

The engineered exosomes may be formulated into pharmaceutical compositions and administered to a subject in need thereof in order to prevent, treat, manage or control a hypothalamus-related disease or disorder in the subject.

Additionally, the engineered exosomes may be loaded with small chemical compounds for targeted delivery of biologically active compounds into cells, tissues and organs. Exemplary small molecules that can be delivered by the disclosed engineered exosomes include, but are not limited to, corticosteroid receptor agonists, such as dexamethasone and cortisol, prednisolone and vamorolone, ulinastatin, chloroquine, eritoran, thalidomide, and didox; inhibitors of TNF-alpha, such as TNF-α inhibitor CAS 1049741-03-8; inhibitors of NF-kB, such as NF-kB activation inhibitor IV-CAS 139141-12-1, KINK-I hydrochloride, Bay 11-7082, pacritinib, R-835, PF-06650833, and AU-4948; inhibitors of PBK; small molecule inhibitors of cytokine signaling; small molecule antagonist of cIAPl/2 and XIAP (X-linked inhibitor of apoptosis protein), such as ASTX660; mitochondria-derived activator of caspase (SMAC) mimetic; AT406 (Debio-1143); inhibitors of IKK Complex, such as SAR-113945, MLN-0415, AS-602868, CHS-828, VGX-1027, teglarinad Chloride (EB-1627; GMX1777, and sulfasalazine; inhibitor of Nuclear Factor Kappa-B Kinase Subunit Gamma (IKKy) inhibitors; IKKc and Tank Binding Kinase I (TBKI) inhibitors; cytokine inhibitors; hormone receptor agonists; hormone receptor antagonists; NF-KB Inducing Kinase (NIK) inhibitors; nuclear receptor agonists; nuclear receptor antagonists; inhibitors of ubiquitin-proteasome system; proteasome inhibitors; NAE (NEDD8 activating enzyme) inhibitors; deubiquitination (DUB) inhibitors; molecules inhibiting nuclear translocation; DNA binding and transcriptional activation of NF-KB, polyphenols-like curcumin, capsaicin, ap1gemn, oleandrin, quercetin, resveratrol, cinnamaldehyde, and epigallocatechin-3-gallate.

Suitable methods for loading purified exosomes include, but are not limited to, transfection, electroporation, expression of fusion proteins that bind the therapeutic during exosome biogenesis, and the like. Exosomes can also be loaded by directly engineering the hypothalamus stem cells to express a recombinant construct as described above.

Suitable diseases or disorders correlated with hypothalamic malfunction that can be prevented, treated, managed or controlled by the administration of engineered stem cells and exosomes produced according to the disclosed method include, but are not limited to, infectious diseases, including pandemic influenza, pandemic respiratory diseases, COVID-19, COVID-19-related diseases, long-term complications of COVID-19, SARS-Co-V2 and variants thereof, and Ebola; functions mediated by vasopressin or antidiuretic hormone, such as high blood pressure, cardiac function, trauma, bleeding disorders, kidney diseases, aberrant plasma osmolarity, diabetes, syndrome of inappropriate antidiuretic hormone (SIDAH). anxiety, PTSD, hemorrhages, septic shock, gastrointestinal bleeding, bedwetting, hyponatremia, hypothyroidism, lethargy, fatigue, loss of appetite, irritability, muscle weakness, spasms or cramps, seizures, and decreased consciousness or coma; functions mediated by the hormone oxytocin, such as postpartum disorders, sexual disorders, autism, anxiety, psychological trauma, post-traumatic stress disorders, and panic disorders; functions mediated by growth hormone-releasing hormone (GHRH) or growth hormone-inhibiting hormone (GHIH), such as acromegaly and growth hormone deficiencies, such as Turner syndrome, Prader-Willi syndrome, brain tumors, acquired growth hormone deficiency (AGHD), delayed puberty or altered sexual development, reduced bone strength, depression, lack of concentration, poor memory; functions mediated by dopamine dysregulation, such as Parkinson's disease, restless leg syndrome, sleep disorders, pain, emotional disorders, muscle cramps, spasms, tremors, aggressive behavior, compulsive behaviors, ADHD, binge eating, gambling addiction, psychosis, schizophrenia, bipolar disorder, Huntington's disease, and Tourette's syndrome; functions mediated by thyrotropin-releasing hormone, thyroid stimulating hormone, thyroxine and triiodothyronine, such as hypothyroidism, thyroid disorders, mood swings, fatigue, lack of sex drive, diarrhea, breast cancer, spinal cord injury, amyotrophic lateral sclerosis, spinocerebellar degenerative disease, regulation of insulin production, function of -cells of the pancreas, thyroid-induced cognitive, neurological and fertility disorders, and immune function; functions mediated by adrenocorticotropic hormone, corticotropin-releasing hormone (CRH) and cortisol, such as aberrations of the hypothalamic-pituitary-adrenal axis, inflammatory diseases, auto-immune diseases, acute respiratory distress syndrome, Cushing's syndrome, and adrenal insufficiency; conditions affecting the pituitary gland, sleep disorders, dysfunctions of the circadian rhythm, hypopituitarism, congenital adrenal hyperplasia, Nelson's syndrome, adrenoleukodystrophy, West syndrome (“infantile spasms”), postorgasmic illness syndrome (POIS), DAVID syndrome, osteoporosis, skin disorders, muscle weakness, amenorrhoea, panic disorder, eating disorders, insomnia, and narcolepsy; functions mediated by gonadotropin-releasing hormone (GnRH), luteinizing hormone, follicle-stimulating hormone or prolactin, prolactin-releasing hormone (PRH) or prolactin-inhibiting hormone (PIH or dopamine), such as menstrual disorders, menopause symptoms and alterations, infertility, in vitro fertilization, endometriosis, prostate cancer, hypogonadotropic hypogonadism, transgender hormone therapy, hot flashes, suppression of spontaneous ovulation as part of controlled ovarian hyperstimulation, hyperandrogenism, hair loss, female androgenetic alopecia, and veterinary medicine; conditions directly or indirectly related to the hypothalamus, such as regulation of circadian rhythms, sleep disorders, mood disorders, physiological functions, fibrillations, Adams-Stokes disease, sinus arrhythmia, Wolff-Parkinson-White syndrome, ventricular tachycardia, slurred speech, decreased motor function, abnormal breathing patterns, fever, hyperthermia, hypothermia, sweating, constipation, irritable bowl disorders, Crohn's disease, ulcerative colitis, pancreatitis, Celiac disease, atherosclerosis, aneurysm, Raynaud's disease, thromboembolism, erythromelalgia, pulmonary embolism, coronary artery disease, carotid artery disease, varicose veins, vasculitis, cancer, substance abuse, and hormone secretion; neurological disorders, such as delirium, including ICU delirium, ventilator-induced delirium, and Covid-19-induced delirium, mental health disorders, including attention deficit hyperactivity disorder (ADHD), adjustment disorder, agoraphobia, Alzheimer's disease, amnesia, amphetamine dependence, anorexia, depression, bipolar disorder, bulimia nervosa, caffeine-induced anxiety disorder, caffeine-induced sleep disorder, cannabis dependence, claustrophobia, cocaine dependence, dementia, depersonalization disorder, dissociative identity disorder, Down syndrome, drug withdrawal, dyslexia, hysteria, impulse control disorder, kleptomania, Korsakoffs syndrome, language disorder, learning disorders, Munchausen syndrome, narcissistic personality disorder, neurodevelopmental disorder, nicotine dependence, nightmare disorder, obsessive-compulsive disorder, oneirophrenia, onychophagia, orthorexia nervosa, paranoia, pedophilic disorder, pyromania, reactive attachment disorder, reading disorder, REM sleep behavior disorder, sadistic personality disorder, selective mutism, separation anxiety disorder, Stendhal syndrome, stereotypic movement disorder, Stockholm syndrome, tardive dyskinesia, vaginismus and voyeuristic disorder; and recreation and preventative health use, including mood enhancement, inducement of pleasure, feelings of wellbeing and vitality, improvement of virility and sexual function in males and females, motivation increase, ability to concentrate on a task, cognitive ability, intelligence and creativity, slowing down aging; cytokine storm, septic shock, multiorgan dysfunction syndrome, septicemia, systemic inflammatory response syndrome, disseminated intravascular coagulation, hypovolemia, stroke, reperfusion injury after stroke, burn trauma, full-thickness burns, post-burn reaction, thermal regulation disorders, severe hypothermia, high altitude illness, acute mountain sickness, high altitude cerebral edema, high altitude pulmonary edema, drug withdrawal symptoms, drug overdose symptoms, drug toxicity and side effects, including side effects such as seizures, hypothermia, hyperthermia, hypotension, hypertension, cardiac dysrhythmias, respiratory depression, ataxia, anxiety, blurred vision, headache, methamphetamine withdrawal symptoms, rhabdomyolysis, excited delirium, malignant hyperthermia, hyperthermia from use or abuse of stimulants such as ecstasy/MDMA, cocaine, methamphetamine, and amphetamines, visual disturbances, eclampsia, pre-eclampsia, epilepsy, seizure disorder, vascular dementia, Lewy body syndrome, fronto-temporal dementia, Huntington's disease, acidosis, alkalosis, anaerobic respiration, cancer, and pregnancy complications.

In additional embodiments, provided herein is a method for identifying a drug that modulates hypothalamus function in a subject in need thereof. The disclosed method comprises (a) transfecting purified human hypothalamus stem cells with a reporter gene operably linked to a transcriptional regulatory element isolated from the subject and regulating expression of the reporter gene; (b) contacting transfected human hypothalamus stem cells with a candidate drug; (c) determining drug effect on transcriptional regulatory element expression by detecting and measuring a signal emanating from the reporter gene and comparing the signal to a signal emanated by the reporter gene prior to contact with the drug, thereby identifying a drug that modulates hypothalamus function in the subject.

In some embodiments, the effect of the drug may be further determined by measuring one or more properties, such as cell viability, proliferation, apoptosis, stern-ness, and differentiation.

Suitable candidate drug include compounds that decrease neuroinflammation, chemotherapeutics, and compounds that alter circadian rhythm, regulate thyroid function or affect obesity, inflammation, infertility or aging.

The subject can be a mammal, such as an animal or a human subject.

The disclosed methods are reliable and quick, and easily scalable for large-scale production of human hypothalamus stem cells and exosomes. In addition, the disclosed human hypothalamus stem cell and exosome compositions are suited to specifically target disorders associated with the neuroendocrine system and the control of behavioral and physiological processes in different subjects, and allow the development of optimal treatment plans for personalized therapy.

The following examples illustrate the disclosed methods for producing and using human hypothalamus stem cells and exosomes thereof according to the present invention. These examples are illustrative only and are not intended to limit the scope of the invention as defined by the claims presented herein.

### EXAMPLES

### Example 1: Identification and Isolation of Human Hypothalamus Stem Cells During Induced Pluripotent Stem Cell Differentiation

It is not known at what stage of pluripotent cell differentiation human hypothalamus stem cells are formed. The aim of this project was to identify and isolate human hypothalamus stem cells at the very intermediate stage of their formation during induced pluripotent stem cell differentiation. The underlying hypothesis was that during the in vitro iPSCs differentiation process, the pluripotent stem cells give rise to progeny that passes through a number of intermediate cell types, only one of which is the hypothalamus stem cell stage. Each progeny contains intermediate stem cells with increasingly restricted differentiation potential as they mature into hypothalamus cells. The goal was, therefore, to identify for the first time the exact stage at which hypothalamus stem cell differentiation occurs.

Pluripotent stem cells grown under conditions that favor the differentiation of mature hypothalamus cells are treated with proteolytic and collagenolytic enzymes to dissociate cell clusters into single cells, and plated in 6 well-coated plates at a density of approximately 1×10⁶ cells/well in E8 medium with 10 μM of Rho-associated protein kinase (ROCK) inhibitor Y27632 overnight. Cell differentiation is then initiated by dual SMAD inhibition using 1 μM LDN193189 and 10 μM SB431542 for 48 hours in DMEM/Fl2 medium supplemented with 1×SLDM supplement (containing 200 ml DMEM/Fl2, 125 mg/ml bovine serum albumin, 27.15 mg/ml sodium bicarbonate, 3.2 mg/ml L-ascorbic acid, 805 μg/ml putrescine, 750 μg/ml D(+)-galactose, 250 μg/ml holo-transferrin, 125 μg/ml catalase, 125 μg/ml L-camitine, 50 μg/ml reduced glutathione, 0.7 μg/ml sodium selenite, 50 μg/ml ethanolamine, 0.1 μg/ml T3 (triiodo-L-thyronine), 1 μg/ml corticosterone, 50 μg/ml linoleic acid, 50 μg/ml linolenic acid, 2.35 μg/ml lipoic acid (thioctic acid), 0.32 μg/ml progesterone, 5 μg/ml retinal acetate, 50 μg/ml a-tocopherol (vitamin E), and 50 μg/ml a-tocopherol acetate), I % PSA (Anti-anti), Ix glutamax, 2.5 μg/ml superoxide dismutase, 4 μg/ml insulin, matrigel and vitronectin.

The cell cultures are then subjected to dual SMAD inhibition with LDN193189, a BMP type I receptor inhibitor of activin receptor like kinases (ALKs) ALK2 and ALK3, and SB431542, a transforming growth factor-beta I (TGF I) of ALK-4, -5 and -7, followed by sonic hedgehog activation with 1 μM smoothened agonist SAG and 1 μM purmorphamine, and by Wnt signaling inhibition with 10 μM IWR-endo from day 2 to day 9 to direct cell differentiation towards ventral diencephalon. The media is regularly replaced every two days. On days 2-8 of culture, small molecule pathway modulators of sonic hedgehog (Shh) (smoothened agonist (SAG), purmorphamine (PMN), and inhibition of Wnt//-catenin signaling (CHIR99021 and IWR-1-endo)) are added to the cultures to stimulate differentiation of the cells towards ventral diencephalon hypothalamic cell identity. Cell cultures are harvested on day 0 and then every day up to day 15 or beyond to determine the presence of hypothalamic stem cells in the culture. Cultures are analyzed for immunophenotype, neurosphere formation and ability to give rise to mature hypothalamus neurons. Hypothalamic cells are identified from day 9 to day 14 or day 15 by detection of the NK2 homeobox I (Nkx2. l) and the homeobox protein orthopedia (Otp; hypothalamic neuron progenitor that specifies neuropeptidergic neurons).

Cells are defined as hypothalamus stem cells if they display one or more cell surface antigens whose presence is characteristic of hypothalamus cells: specifically Nkx2.lhi, Raxhi, Soxl¹⁰, as well as mouse neuronal stem cell antigens Sox2+ and Bmi-1+. Hypothalamus stem cells are further defined as being nestin+, Musashil+ and Cxcr4+. A gradient is observed, where expression of hypothalamus markers gradually increases beginning on day 0 and over time, whereas expression of neuronal stem cell markers reaches a pick and thereafter declines. Hypothalamus stem cells are identified and isolated when they reach maximal expression of hypothalamus markers and neuronal stem cell markers, while retaining their ability to self renew by forming neurospheres and differentiate into mature hypothalamus cells.

### Example 2: Differentiation of Human Hypothalamus Stem Cells into Mature Hypothalamus Neurons

To determine whether human hypothalamus stem cells are capable of developing into mature hypothalamus neurons, day 9 to day 15 stem cell growth is inhibited by addition of 10 μM DAPT and caudalization with 0.01 μM retinoic acid. On day 14, the cells are treated with proteolytic and collagenolytic enzymes to dissociate cell clusters into single cells, and the cells are re plated onto laminin-coated plates and maintained in medium containing 10 ng/ml brain-derived neurotrophic factor BDNF until day 40.

Detection of hypothalamic markers Otp, Rax, neuropeptide Y (NPY, a secreted neuropeptide of the orexigenic NPY neurons), cocaine amphetamine regulated transcript (CART, a neuropeptide produced by anorexigenic CART neurons), a-melanocyte stimulating hormone (a-MSH, a bioactive product of POMC producing neurons), neuropeptide Y receptor Y2 (NPYR), ghrelin receptor (GhrR, ghrelin response receptors present in ARC neurons), and melanin concentrating hormone (MCH, orexigenic hypothalamic peptide found in hypothalamic MCH neurons) indicates that human hypothalamus stem cells developed into mature hypothalamus neurons.

### Example 3: Neurosphere Formation

To obtain primary cultures of hypothalamus stem cells, the hypothalamus and hippocampus are dissected from human cadavers or non-human animals, cut into small pieces of approximately 1 mm, and enzymatically digested for 30 min at 37° C. The cells are centrifuged, suspended in neurobasal medium containing 0.24% L-alanine, L-glutamine supplement, 2% B27 without vitamin A, 10 ng/ml EGF, 10 ng/ml bFGF, and 1% penicillin-streptomycin, and seeded in ultralow adhesion 6-well plates for one week. Neurospheres are then collected by centrifugation, enzymatically digested into single cells, passaged and maintained in neurosphere culture until use.

### Example 4: Differentiation of Human Embryonic Stem Cells and Induced Pluripotent Cells into Neurons

Human embryonic stem (ES) cells or human induced pluripotent (hPS) cells are cultured on a layer of feeder cells or in a feeder-free system with no differences in hypothalamic differentiation. hPS cells are cultured in 6-well plates in the presence of mouse embryonic fibroblasts (MEF). The cells are then seeded on matrigel-coated plates (feeder-free) to initiate neuron differentiation once each well reaches 95-100% confluence. The hPS cells have uniform shape and actively proliferate upon starting differentiation. SB 431542 and LDN 193189 are added from day 1 to day 8 to inhibit transforming growth factor beta (TGF) and bone morphogenetic protein (BMP) signaling and promote neuron differentiation from human ES/iPS cells. Sonic hedgehog (SHH) signaling molecule and Purmorphamine (PMA) are also added from day 1 to day 8 to induce ventral brain development and NKX2. l expression. Further inhibition of Notch signaling is obtained by addition of DAPT from day 9 to day 15 to increase NKX2. l and activity-regulated cytoskeleton-associated protein (Arc) expression for neuron precursor formation.

6-well plates are seeded on day 0, and from day 0 to day 15 a cell culture is harvested every day. Hypothalamic stem cells are identified between day O and day 15 of culture by marker detection, immunophenotype analysis, visualization of neurosphere formation, and by detecting the presence of mature hypothalamus neurons in the culture, as described above. Specific marker detection includes detection of Nkx2. l hi, Raxhi, Soxl¹⁰, and neuronal stem cell antigens Sox2+ and Bmi-1+. Hypothalamus stem cells may be further defined as being nestin+, Musashi f and Cxcr4+. A gradient is observed, where expression of hypothalamus markers gradually increases beginning on day O and over time, whereas expression of neuronal stem cell markers reaches a pick and thereafter declines. Hypothalamus stem cells are identified and isolated when they reach maximal expression of hypothalamus markers and neuronal stem cell markers, while retaining their ability to self renew by forming neurospheres and differentiate into mature hypothalamus cells.

### Example 5: Exosome Formation

Hypothalamus stem and progenitor cells are obtained as described above. Exosomes secreted into the culture medium are then purified by differential centrifugation at 4° C. overnight to remove impurities and generate a medium with a lesser content of exosomes. The cells are cultured in the exosome-impoverished medium for two days, after which the medium is collected, centrifuged to remove cells, and exosomes are immediately isolated at 4° C. The medium is centrifuged and filtered to remove debris, and the exosomes in the filtered medium are isolated by differential centrifugation. An analogous volume of uncultured medium is subjected to the same purification procedures and used as control. Isolated exosomes are analyzed by different approaches.

In a first approach, immunoblot analysis of exosomal protein markers is performed. Exosomal and cellular proteins are loaded and separated by SDS-PAGE, followed by western blotting analysis with primary antibodies: mouse anti-TSG101, rabbit anti-CD81, rabbit anti-AGO2, rabbit anti-HSP90Bl, mouse anti-GM130, and rabbit anti-Cycl. The exosomes are then detected by silver staining. In a second approach, flow cytometry is performed. Purified exosomes are incubated with latex beads for 15 minutes, adjusted to a final volume, and rotated at room temperature for 2 hours. Glycine is added and after incubation, beads are collected by centrifugation, resuspended and stained by FACS using FITC-conjugated anti-CD8 l antibody. In a third approach, nanoparticle-tracking analysis of the purified exosomes is performed. In a fourth approach, the exosomes are analyzed by density-gradient ultracentrifugation analysis. The isolated exosomes are resuspended in 2.5M sucrose solution (20 mM HEPES, pH 7.4), plated on a continuous sucrose gradient from 2M to 0.25M, and ultracentrifuged overnight at 4° C. Different fractions are collected, each fraction recovered by ultracentrifugation and analyzed by immunoblot and real-time PCR. In a fifth approach, the exosomes are analyzed by pull-down assay: anti-CD8 l and isotype-control antibodies are coated onto Protein G beads, the exosomes are re-suspended in PBS containing 3 mg/ml BSA, and incubated with antibody-coated beads overnight with rotation at 4° C. The beads are subsequently washed with the same buffer, and the isolated exosomes are analyzed by real-time PCR. In a sixth approach, exosomal total protein and RNA are measured.

### Example 6: Collection of Conditioned Media and Purification of Exosomes

Conditioned media (CM) from cells cultured as described above is harvested every 48 hours and filtered through a 0.22 m membrane to remove cell debris and larger vesicles. CM is collected and stored in batches at −20° C. until use. The CM batches are then thawed, combined and subject to PEG precipitation by incubation with PEG 6000 for 8-12 hours at 4° C., followed by differential centrifugation for 30 minutes at 1,500 g to obtain exosome pellets. The pellets are resolved in 1 ml 0.9% NaCl, washed in a total volume of 45 ml 0.9% NaCl for 12 hours at 4° C., and separated by ultracentrifugation for two hours at 100,000 g. The pellets are again resolved in 1 ml 0.9% NaCl, diluted in with 0.9% NaCl to reach the required concentrations, and stored as 1 ml aliquots at −80° C. until use.

### Example 7: Quality Control Assessment of Exosomes

The quality of the exosomes obtained as described above is assessed by determining the concentration of protein in each aliquot. For each aliquot, 10 g of exosome-containing fraction is analyzed by Western blots using antibodies that recognize exosome-specific marker proteins: Tsgl01, CD63, CD81 and/or CD82. The concentration and size of the exosomes is additionally assessed by nanoparticle tracking analysis, and qPCR is used to determine the content and, optionally, the bioactivity of the exosomes when administered to cells.

### Example 8: Purification of Exosomes by Differential Centrifugation

Conditioned media (CM) from cells cultured as described above is harvested every 48 hours and filtered through a 0.22 m membrane to remove cell debris and larger vesicles. Differential ultracentrifugation is used to separate the exosomes by their vesicle size and sedimentation properties. Sequential centrifugation steps with increasing force of centrifugation deplete the conditioned medium from large particles and/or vesicles with high sedimentation rates. A final ultracentrifugation (UC) step sediments small vesicles or exosomes, and leaves the smaller proteins in the supernatant.

The cell culture medium is replaced with exosome-depleted medium, which is obtained by centrifuging stem cell medium at 100,000 g for at least 17 hours, and incubated for 48 hours. The exosomes are then purified from the conditioned medium by differential UC. Cell debris is pelleted at 300 g for 10 minutes. Larger vesicles are pelleted at 10,000 g for 30 minutes, and the supernatant is filtered through a 0.2 m membrane. The exosomes are then pelleted at 100,000 g for 90 minutes using 70 ml polycarbonate bottles and Type 45 Ti rotor. Exosome pellets are washed once in 1 ml sterile PBS and centrifuged for 90 minutes at 100,000 g in a tabletop ultracentrifuge using a TLA-110 rotor.

### Example 9: Purification of Exosomes by Tangential Flow Filtration

Conditioned media (CM) from cells cultured as described above is harvested every 48 hours and filtered through a 0.22 m membrane to remove cell debris and larger vesicles. Exosomes are isolated and purified from three-dimensional cell cultures by growing the cells on the surface of spherical support matrix beads with continuous stirring. A pump circulates the conditioned culture medium through membranes or filters to remove small particles and maintain larger particles in optimal concentration. In particular, the conditioned cell culture supernatant is first passed through a 200-nm pore size membrane to remove large vesicles and particles.

The filtered conditioned medium is subjected to tangential flow filtration using a hollow fiber filter with a 500-kDa molecular weight cutoff (MWCO). A feed flow rate of 120 ml/min, trans-membrane pressure <3.5 psi, and a cross-flow rate >10:1 is maintained throughout the filtration operation. The conditioned medium is concentrated 9-fold and then replaced with 6× volume of PBS, by continuously feeding the system with PBS to replace the loss of permeate. The isolated exosomes are filtered through a 0.2 m membrane and stored in 0.1M sucrose in polyethylene terephthalate glycol (PETG) bottles at −80° C.

### Example 10: Exosome Transfection

Exosomes produced as described above are engineered to express foreign nucleic acids, including siRNAs. 4.5×10¹⁰ exosomes are co-incubated with 1 nmol of siRNA at 37° C. for 1 hour in 500 μl of PBS to obtain a loading mixture. The exosome-siRNA mixture is centrifuged at 100,000 g for 90 minutes, and the supernatant containing unloaded siRNA is removed. The pellets are suspended in 500 μl of PBS to measure fluorescence, or in 300 μl of neural medium to treat primary neurons. To quantify loading of Cy3-labeled siRNA, a 200 μL aliquot is taken from the suspended exosome pellet or from the supernatant. Fluorescence is measured at 550 nm excitation, 570 nm emission. The percent loaded siRNA is calculated as pellet/(pellet+supernatant). The siRNA copy number per exosome is estimated as: (percent of loaded siRNA)×(amount of siRNA initially mixed with exosomes [mol])×(Avogadro number)/(number of exosomes initially mixed in).

For exosome transfection with microRNA, 4.5×10¹⁰ exosomes are co-incubated with 1 nmol of miRNA at 37° C. for 1 hour in 500 μl of PBS to obtain a loading mixture. The exosome-miRNA mixture is centrifuged at 100,000 g for 90 minutes, and the supernatant containing unloaded miRNAs is removed. The pellets are suspended in 500 μl of PBS to measure fluorescence, or in 300 μl of neural medium to treat primary neurons. To quantify loading of Cy3-labeled miRNA, a 200-1 aliquot is taken from the suspended exosome pellet or from the supernatant. Fluorescence is measured at 550 nm excitation, 570 nm emission. The percent loaded miRNA is calculated as pellet/(pellet+supernatant). The miRNA copy number per exosome is estimated as: (percent of loaded miRNA)×(amount of miRNA initially mixed with exosomes [mol])×(Avogadro number)/(number of exosomes initially mixed in).

### Example 11: Isolation of Human Hypothalamus Stem Cells and Exosomes from Humans

Hypothalamus cells are isolated from the hypothalamus region of the brain and from the median eminence of the third ventricle in human adult subjects and in young infants and children less than two years old, following brain resection surgery. Hypothalamus cells are also isolated from the same regions in cadavers.

The cells are placed in basal media supplemented with glutamine, insulin, nitrogen, B27 supplement, and growth factors, including EGF, FGF-2, IGF-1, BNDF, NT-3, DKKI, GNDF, laminin, and BMPR1a-Fc, to stimulate neural stem cell growth.

Cells that express both hypothalamic and stem cell markers, including Nestin, Sox-2 and Musashi, are selected, and those expressing a neural stem cell immunophenotype are separated using antibodies to the antigens ABCG2, CD133, CXCR4, FGF R4, Frizzled-9 Glutl, SSEA-1, Notch-I and Notch-2.

The isolated cells are then immortalized, and human hypothalamus stem cell exosomes are collected as described above.

### Example 12: Identification of Drugs that Decrease Neuroinflammation for the Treatment of Diseases of the Hypothalamus

To identify drugs that decrease neuro-inflammation, the immortalized hypothalamus cells obtained as described above are transfected with a reporter gene operably linked to NFKB p50/p65, a transcriptional regulatory element, which is isolated from a target organism and regulating the expression of the reporter gene.

The transfected immortalized hypothalamus cells are contacted with a candidate drug, and the signal emanating from the reporter gene is detected and compared to the signal emanated by the reporter gene before exposure of the cells to the drug, to obtain the drug response profile.

### Example 13: Identification of Drugs that Alter the Circadian Rhythm for the Treatment of Sleep Disorders and Other Diseases of the Hypothalamus

To identify drugs that alter the circadian rhythm, the immortalized hypothalamus cells obtained as described above are transfected with a reporter gene operably linked to the promoter regions of the period (per) and timeless (tim) genes, or to the NR2f6 gene, and contacted with a candidate drug, to determine the effect of the drug on sleep disorders and the circadian rhythm in a subject.

### Example 14: Identification of Drugs for the Treatment of Thyroid Disorders

To identify drugs for the treatment of thyroid disorders, such as obesity and impaired metabolism, the immortalized hypothalamus cells obtained as described above are transfected with a reporter gene operably linked to the promoter regions of the thyroid hormone receptor gene, and contacted with a candidate drug, to determine the effect of the drug on thyroid disorders in a subject.

### Example 15: Identification of Drugs that Correct Human Hypothalamus Function for the Treatment of Diseases of the Hypothalamus

To identify drugs that correct human hypothalamus function, the immortalized hypothalamus cells obtained as described above are transfected with a reporter gene operably linked to one or more of the HMX2 (Nkx5-2), SIMI (bHLHel4), PITX2 (ARPI), SOX14 (SOX28), HMX3 (Nkx5-l), SIX6 (OPTX2), DMBXI (OTX3), EBF3 (COE3), NKX2-l(BCH), CITED I (MSGI), ISLI (Isl-I), and LHX5 genes.

The transfected immortalized hypothalamus cells are contacted with a candidate drug, and the signal emanating from the reporter gene is detected and compared to the signal emanated by the reporter gene before exposure of the cells to the drug, to obtain a personalized drug response profile for a human subject.

### Example 16: Identification of Drugs for the Treatment of Diseases of the Hypothalamus

To identify drugs for the treatment of hypothalamus disorders, such as obesity, inflammation, infertility and aging, hypothalamus stem cells obtained from a human subject as described above are plated in 96-well plates. Each well is contacted with a candidate drug and a biological property selected from cell viability, proliferation, apoptosis, stem-ness, and differentiation is measured by one or more techniques including XTT, MTT, BrdU, EdU, WST-1, luminescent ATP, Ki67, CFSE, trypan blue, Live/Dead, Annexin V, mitochondrial membrane potential, cytochrome C, JC-I dye, biotracker mitochondria dye, mitochondrial apoptosis detection, mitochondrial depolarization stress damage, Caspase-3 dye, Caspase-3,7, Caspase-8, Caspase-9, Caspase activity, TUNEL, DNA fragmentation detection, cell death detection, H2A.X phosphorylation, immuno-fluorescent staining, neurosphere formation, and self-renewal.

### Example 17: Scaling Up Hypothalamus Stem Cell Production

Pluripotent cells are grown into aggregates in suspension and then differentiated into hypothalamus stem cells as described below.

High quality human pluripotent stem cell lines are cultured in 2D and passaged using EDTA in phosphate buffered saline onto a non-tissue culture treated vessel devoid of matrix proteins in basal media supplemented with 0.12 ng/ml transforming growth factor (rh TGF) and 4 ng/ml fibroblast growth factor (rh bFGF), 2 mM L-glutamine, 0.1 mM beta-mercaptoethanol, I % non-essential amino acid stock, and, optionally, with 1,000 to 3,000μ/ml rh LIF, 25 ng/ml IL-6, 25 ng/ml sIL-6R, and 15% knock-out serum replacement. Initial cultures are supplemented with Y-27632 to promote survival. The vessel is placed in a C02 incubator at 37° C. with rotation at 30-100 rotations per minute.

The pluripotent stem cell lines are expanded, and the cultures are passaged when the density of the cells reaches 1-2×10⁶ viable cells/ml. several times.

To induce differentiation, the media is replaced with differentiation basal media supplemented with glutamine, insulin, Ix B27, 1 μM LDN193189 and 10 μM SB431542, and after 48 hours the media is further supplemented with 1 μM SAG, 10 μM IWR and 1 μM PMN from day 2 to day 9, with several passages. After 9 days of culture, the media is replaced with differentiation basal media supplemented with glutamine, Ix N2, Ix B27, insulin, 10 μM DAPT, and 0.01 μM retinoic acid.

Cell cultures are harvested between day 9 and day 15 post-differentiation induction.

### Example 18: Isolation of Hypothalamus Stem Cell Exosomes from Biological Fluids

Samples of biological fluids, including cerebral spinal fluid, serum, plasma, amniotic fluid, nasal swab, are obtained from a subject and diluted with phosphate buffered saline. The samples are then treated with beads coated with antibodies that recognize hypothalamus stem cell exosomes, including antibodies to SLC 1 8A2, SLC18A3, SLC17A6, SHISAL2B, BRS3, DLK-1, GABRE, GOLTIA, TMEM114, ABCG2, CD133, CXCR4, FGF R4, Frizzled-9, Glutl, SSEA-1 Notch-I and Notch-2.

The antibody-coated exosomes are washed with phosphate buffered saline containing exosome-depleted serum and detergent to prevent non-specific binding, and the exosomes conjugated with the antibodies are isolated and purified.

The exosomes thus obtained are combined with biomarkers for prognostics and diagnostic determinations, such as prenatal diagnostics, or engineered for targeted therapy or research as described above.

### Example 19: Treatment of Chronic Inflammation by Delivery of Hypothalamus Stem Cell Exosomes Loaded with NFkappaB Inhibitor

Purified exosomes produced as described above are loaded with a therapeutic that suppresses NFkappaB activation by electroporation and delivered across the blood brain barrier of a subject suffering from chronic inflammation. The therapy results in gradual decrease of chronic inflammation, until complete healing.

### Example 20: Human Hypothalamus Stem Cells

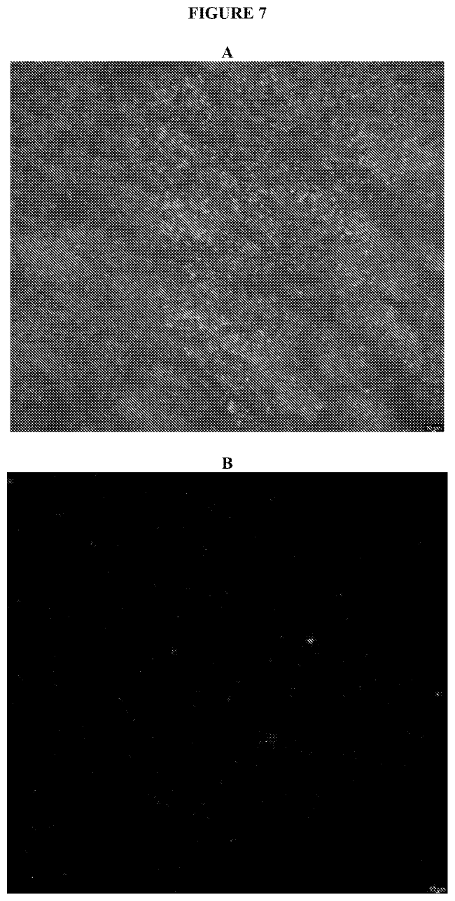

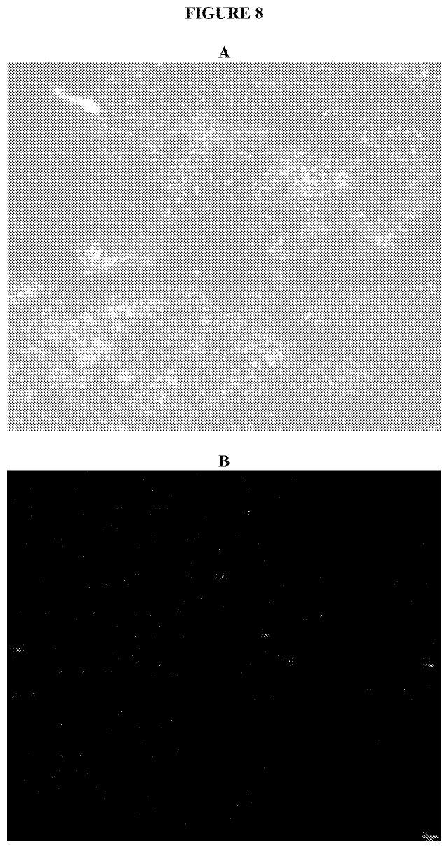

Human hypothalamus stem cells created by differentiation of induced pluripotent stem cells express markers of both hypothalamus cells (FIG. 7) and of neural stem cells (FIG. 8). BYS0I 12 induced pluripotent stem cells were obtained from ATCC. BYS0I 12 cells were cultured in mTesr Plus medium on plates pre-coated with hESC-qualified Matrigel. Matrigel was aliquoted m volumes per manufacturer recommendations for the dilution factor, and diluted in 25 ml of PBS or DMEM/Fl2 without serum. Tissue culture plates were treated with dilute Matrigel for at least one hour using a volume of 1 ml/well of a 6-well plate, or 6 ml/100 mm dish. BYS0I 12 cells were passaged as aggregates using enzyme-free dissociation methods based on EDTA.

To initiate differentiation, BYS0I 12 cells were dissociated enzymatically, seeded in a tissue culture-treated vessel at a density of 70,000 cells/cm² and grown overnight in mTesr Plus medium containing the ROCK inhibitor Y-27632. The next day media was changed to differentiation media A Cells were cultured in this media for 2 days (48 hours), with daily media changes. Cells were then cultured in differentiation media B for a time period between 4 and 7 days with daily media changes. Cells were then cultured in differentiation media C until hypothalamus stem cells were produced. Hypothalamus stem cells were observed between day 9 of culture and day 15 of culture and exosomes were collected from cell culture supernatant during this time. Once pluripotent are differentiated into hypothalamus stem cells, hypothalamus stem cells are found in culture for several days, weeks and in some cases months under self-renewal conditions. In the absence of growth factors that promote the differentiation of hypothalamus stem cells, the hypothalamus stem cells can be maintained as hypothalamus stem cells in culture indefinitely in differentiation media C, and immortalized by exogenously expressing an immortalizing oncogene such as hTERT or SV40 largeT antigen.

The composition of differentiation media A is basal media supplemented with Ix B27 plus, Ix glutamax, cl-glucose, insulin 4 ug/ml, super-oxide dismutase 2 ug/ml, 1 uM LDN193189 and 10 uM SB431542. The media is optionally supplemented with recombinant sonic hedgehog protein, 10 uM IWR-lendo, 1 uM smoothened agonist SAG or 1 uM purmorphamine (PMN).

The composition of differentiation media Bis basal media supplemented with Ix B27 plus, Ix glutamax, cl-glucose, insulin 4 ug/ml, super-oxide dismutase 2 ug/ml, 10 uM IWR-lendo, 1 uM purmorphamine (PMN) and 1 uM SAG. In some experiments the amount of B27 was reduced to 0.5× and N2 supplement was added at a concentration of 0.5×.

The composition of differentiation media C is basal media supplemented with Ix B27 plus, Ix glutamax, cl-glucose, insulin 4 ug/ml, super-oxide dismutase 2 ug/ml, and 10 uM DAPT. In some cases the amount of B27 plus was reduced to 0.5× and N2 supplement was added at a concentration of 0.5×. In other cases I x B27 plus was replaced with IXN2.

### Example 21: Human Hypothalamus Stem Cell Exosome Purification and Characterization

Hypothalamus stem cell exosomes were purified from hypothalamus stem cell conditioned medium using ultrafiltration followed by size exclusion separation. Standard laboratory protection equipment (gloves, coat, goggles, and mask) were used on all steps of sample preparation and analysis to prevent samples contamination with dust particles. Conditioned cell culture media from hypothalamus stem cells was collected after 48 hours of culture. The media was frozen until further analysis. The conditioned medium was thawed on ice on the day of analysis. For protease inhibitor (PI) preparation, 1 tablet of protease inhibitor was sufficient for 10 ml of media. 10× stock solution of protease inhibitors (PhosStop and Complete PI cocktail, 1 tablet each) was prepared by dissolving 2 tablets of each in 2 ml of 0.22 m-filtered PBS. Then, 1.5 ml of PI solution was added to 15 ml of thawed conditioned cell culture media.

Ultrafiltration was performed to remove proteins and low molecular compounds and to concentrate the sample for loading on the size exclusion column. Conditioned medium with protease inhibitors was quickly centrifuged at 1000×g to remove large debris and cell fragments. The ultrafiltration devices containing 100 kDa molecular weight cutoff (MWCO) PES membrane were rinsed with PBS before use by adding 1 ml of PBS to the filter and spinning the device at 3,000×g for 5 min. Any remaining retentate, as well as the eluate, were discarded. Conditioned medium was loaded into pre-rinsed filter device and centrifuged at 3000 g for 30 minutes until the retentate volume reached 100 μl. The retentate was transferred to new tubes. Membranes were washed with 0.2 ml of PBS by pipetting up and down at least 10 times. The wash was repeated two times. The wash solutions were combined with conditioned medium retentate to a final volume 0.5 ml.

Size Exclusion Separation was then performed. Size exclusion separation resolves particles by size. Larger particles elute first on the column, followed by proteins and small molecular weight compounds. Izon 35 nm qEV original column was washed with 20 ml of freshly filtered PBS. With cap closed, the buffer from the top of the column was removed and 500 μl of the sample was loaded. Cap was opened immediately and 0.5 ml fractions were collected. The column was not allowed to dry out at any time, and fresh PBS was added at the top when needed to maintain the flow. First 6 fractions (3 ml)-void volume, were discarded. Exosome fractions 7, 8 and 9 were collected and pooled together. The exosome fractions were concentrated by centrifugation. A total of 50 μl was recovered after centrifugation and transferred into a new tube. The filter membranes were rinsed with 100 μl of PBS by pipetting up and down 10 times. The wash buffer was combined with the exosome retentate and washed one more time. The total volume of the exosome fraction was 250 μL. Protein concentration was measured.

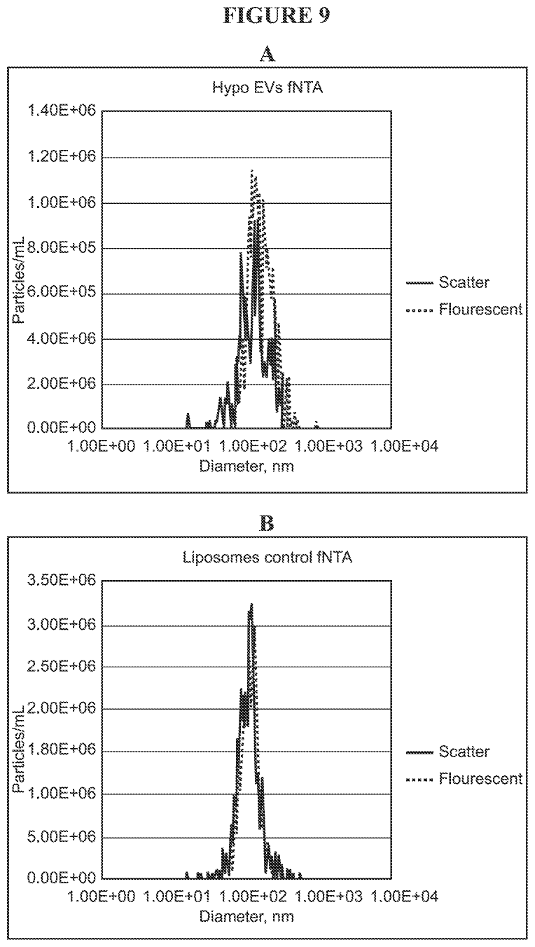

Nanoparticle tracking analysis (NTA) was used to quantify the number of hypothalamus stem cell exosomes and to determine their size (FIG. 9). NTA is a technique for visualization and analysis of dilute aqueous particles solutions. Particles are visualized as they scatter light of the laser beam passing through the sample cell. Particles with size under 1000 nm freely move in solution under Brownian motion. Visualized particles tracks are recorded by camera. Track length traveled by particles per unit of time is analyzed by software and allows determination of a size, size distribution profile and concentration of particles with a diameter of approximately 30-1000 nm. Particle size is calculated to a sphere equivalent hydrodynamic radius through the Stokes-Einstein equation.

Fluorescent NTA technique involves labeling of intact exosomal membrane with a fluorescent dye and then performing the analysis in scatter and fluorescent modes. This technique allows exclusion of contaminant particles, such as protein aggregates, lipoproteins, etc. and assess purity of exosome samples. The analysis shown in FIG. 9 was performed by fluorescent NTA equipped with 520 nm laser, 550 nm long pass cut off filter and sCMOS camera. DI water was filtered on the day of analysis through 0.22 m syringe filter and its purity confirmed by NTA prior to the study. Instrument calibration was performed with 100 nm polystyrene fluorescent beads. For exosome labeling, 12 μl of reaction buffer were mixed with 2 μl of dye and 5 μl of sample. The mixture was vortexed for 15 seconds and samples were incubated at RT for 10 minutes. Liposomes were used as labeling control: 1 μl of liposomes was mixed with 12 μl of reaction buffer and 2 μl of dye. Dilutions were made by mixing DI water filtered through 0.2 μm syringe filter with corresponding volumes of a sample.

### Example 22: Visualization of Human Hypothalamus Stem Cell Exosomes by Electron Microscopy

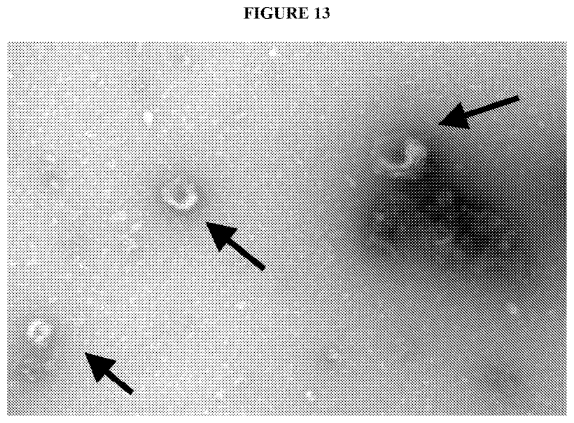

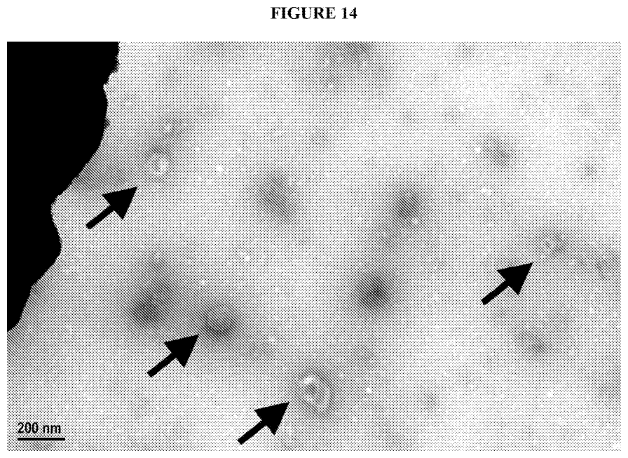

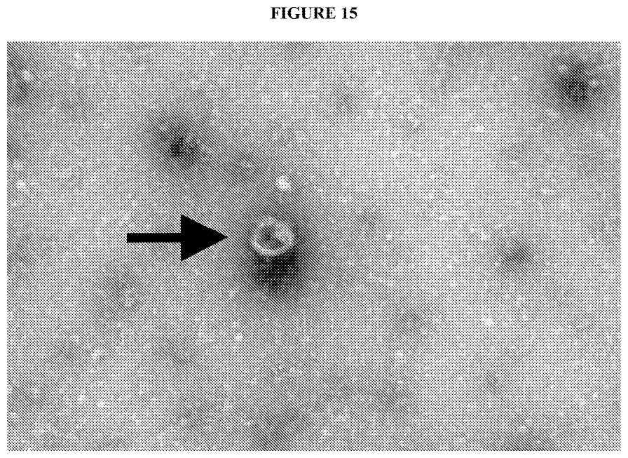

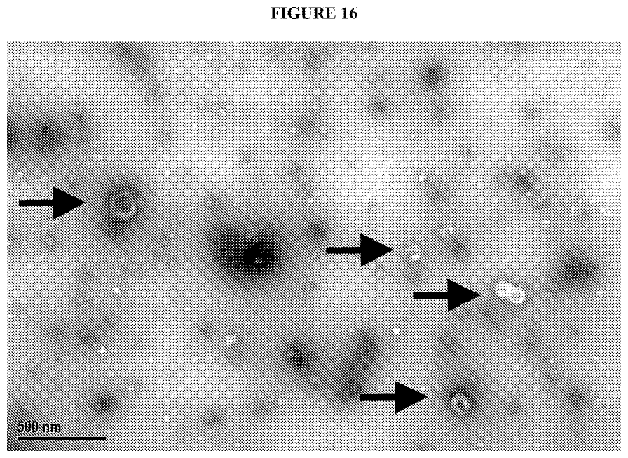

Transmission electron microscopy was used to visualize the human hypothalamus stem cell exosomes (FIGS. 13-16). Copper carbon grids were glow-discharged immediately prior to loading the samples. Sample was processed undiluted. Grid was floated on 10 μl sample drop for 15 minutes, washed twice with water by floating on the drop of water for 30 seconds, and negatively stained with 2% uranyl acetate by floating on the drop of stain for 30 seconds. The grid was blot dried with Whatman paper and imaged by electron microscope.

### Example 23: Capillary Western Blots Confirm that Human Hypothalamus Stem Cell Exosomes Express Proteins Associated with Exosomes

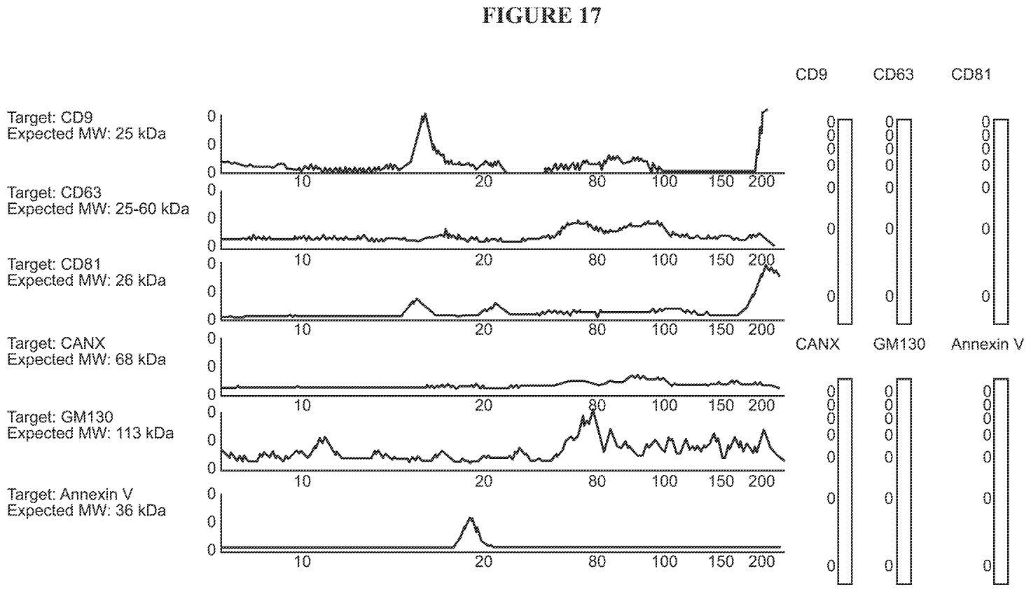

Purified hypothalamus stem cell exosomes were analyzed for expression of 4 positive controls—CD9, CD63, CD81 and Annexin V—and 2 negative controls—GM130 and CANX—by capillary western blot (FIG. 17). All four positive controls showed corresponding peaks. GM130 was not detected in the sample, while some levels of CANX were observed. Samples and reagents were loaded into an assay plate and then the samples were loaded into the capillary automatically and separated by size as they migrated through a stacking and separation matrix. The separated proteins were then immobilized on the capillary wall by proprietary, photoactivated capture chemistry. Target proteins are identified using a primary antibody and immunoprobed using an HRP-conjugated secondary antibody and chemiluminescent substrate. The resulting chemiluminescent signal is detected and quantitated.

### Example 24: Hypothalamus Stem Cell Exosomes Decrease Inflammation in Human Cells

Inflammation in the human brain has been associated with neurodegenerative disease. To validate the utility of human hypothalamus stem cell exosomes the human astrocyte cell line (CHLA-03-AA cells) was treated with inflammatory cytokines to simulate inflammation and then treated with hypothalamus stem cells or vehicle control. Gene expression of genes associated with inflammation was then assessed. CHLA-03-AA cells were grown in DMEM:Fl2 Medium with 20 ng/ml human recombinant EGF, 20 ng/ml human recombinant basic FGF, and B-27 Supplement to a final concentration of 2% (v/v). CHLA-03-AA cells were seeded at 50,000 cells/cm² into the wells of the 24-well carrier plates (1.9 cm²/well). 10 ng/ml recombinant human IL-I and 10 ng/ml recombinant human TNF-α was added to stimulate inflammation. Mock-exosomes isolated from a no-cell negative control or 50 ug of hypothalamus stem cell exosomes was added to the cultures. RNA was purified and 1 ug of RNA was used for reverse transcription. The ability of human hypothalamus stem cell exosomes to abate transcription of inflammatory genes was measured by qPCR using the following primers:

TNF-alpha
FWD:
CTCTTCTGCCTGCTGCACTTTG,
RVS:
ATGGGCTACAGGCTTGTCACTC;
Interleukin-6
FWD:
GACAGCCACTCACCTCTTCAG;
RVS:
TTCTGCCAGTGCCTCTTTGCTG;
Interleukin-I beta
FWD:
CCACAGACCTTCCAGGAGAATG,
RVS:
GTGCAGTTCAGTGATCGTACAGG;
NFkappaB1
FWD:
GCAGCACTACTTCTTGACCACC,
RVS:
TCTGCTCCTGAGCATTGACGTC;
TGFB3
FWD:
CTAAGCGGAATGAGCAGAGGATC,
RVS:
TCTCAACAGCCACTCACGCACA;
Beta actin
FWD:
CACCATTGGCAATGAGCGGTTC,
RVS:
AGGTCTTTGCGGATGTCCACGT.

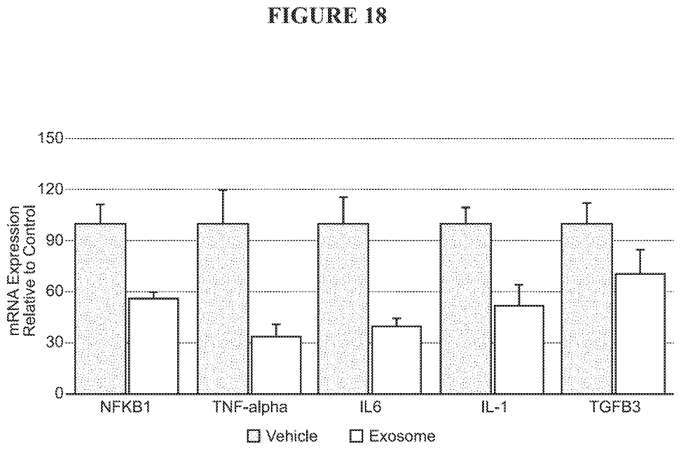

FIG. 18 shows that human hypothalamus stem cell exosomes are able to reduce the expression of inflammatory genes in human cells.

### Example 25: Hormones can be Loaded onto Hypothalamus Stem Cell Exosomes

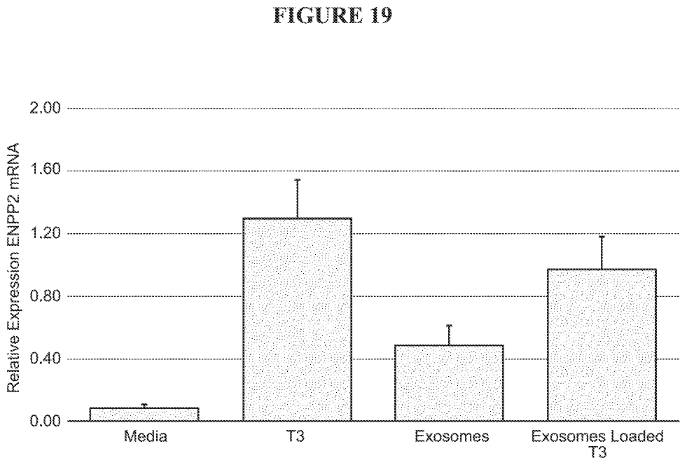

The thyroid hormone receptor ligand T3 was loaded into hypothalamus stem cell exosomes that were then administered to human cells to determine whether loading of bioactive molecules into hypothalamus stem cells causes biological effects. The gene ectonucleotide pyrophosphatase/phosphodiesterase 2 (ENPP2) is a gene that is activated upon activation of the thyroid-hormone receptor by the ligand T3. FIG. 19 shows that transcription of the gene ENPP2 is activated following administration of exosomes loaded with T3, similar to treatment with T3.

Human SK-N-AS neuroblastoma cells were obtained from ATCC and grown in DMEM supplemented with 0.1 mM Non-Essential Amino Acids (NEAA) and 10% exosome-depleted fetal bovine serum that was charcoal-stripped the night prior to incubator with T3. Human hypothalamus stem cells were loaded with T3 by sonication using six cycles of 30 s on/off for a total of 3 min with 2 min cooling period in a dismembrator with a 0.25″ tip at 20% amplitude. Exosomes were allowed to recover for 60 minutes by incubation at 37° C. Unincorporated siRNA was removed by purifying exosomes using an exosome spin column (MW 3000). Some exosomes are loaded by electroporation at a voltage of 750 with 10 pulses of 20 ms, or by passive loading by incubation of exosome and drugs in a I:I ratio.

SK-N-AS cells were treated either with media only mock-exosome negative control, 100 nM T3, 50 ug hypothalamus stem cell exosomes, or 50 ug hypothalamus stem cell exosomes loaded with T3. Cells were incubated for 24 hours before RNA purification. 1 ug of RNA was used for reverse transcription. The ability to activate the thyroid hormone receptor was measured by qPCR. The hENPP2 gene was amplified using the primer sequences FWD: ACTCCGTGAAGGCAAAGAGA, and RVS: CAAGATCCGGAGATGTTGGT. The housekeeping gene cyclophilin A was amplified with the primer sequences FWD: GGCAAATGCTGGACCCAACAC and RVS: TGCCATTCCTGGACCCAAAGC.

### Example 26: siRNA is Loaded into Hypothalamus Stem Cell Exosomes

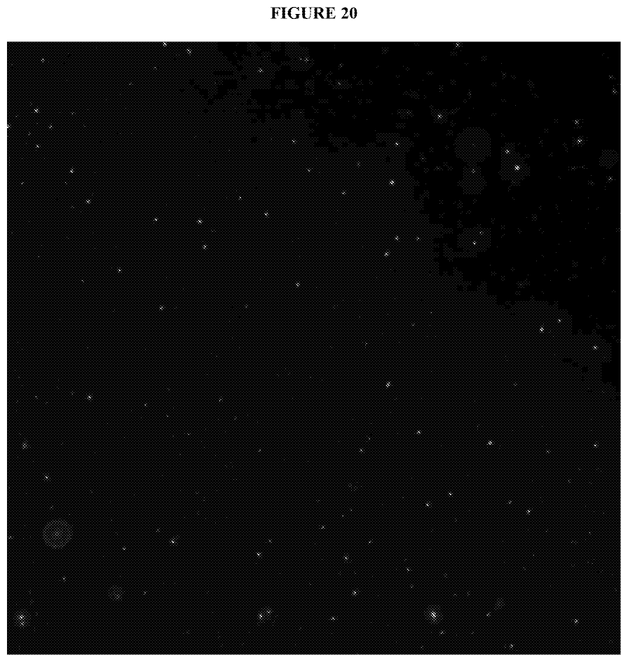

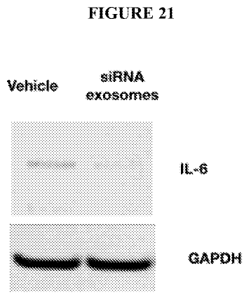

Human hypothalamus stem cells were engineered to express exogenously added siRNA molecules. Human hypothalamus stem cells were loaded with control siRNA or siRNA molecules to IL-6 by electroporation at a voltage of 750V with IO pulses of 20 ms. Electroporation cuvettes were pre-chilled on ice for 30 minutes prior to electroporation. 10 ug of exosomes in PBS were mixed with 0.47 ug of the respective siRNA in a total volume of 150 ul with citric acid buffer. Following electroporation exosomes were incubated at 37° C. for 30 minutes to allow recovery of the exosomal membrane. Unincorporated siRNA was removed by purifying exosomes using an exosome spin column (MW 3000). FIG. 20 shows siRNA incorporation as indicated by fluorescence of hypothalamus stem cell exosomes. 50 ug of siRNA loaded exosomes or vehicle control was then administer to CHLA-03-AA cells treated with 10 ng/ml recombinant human IL-I and 10 ng/ml recombinant human TNF-α was added to stimulate inflammation. Cells were lysed in RIPA buffer and protein expression of IL-6 was measured by Western Blotting. FIG. 21 shows silencing of IL-6 following treatment with hypothalamus stem cell exosomes loaded with IL-6 siRNA.

In addition to electroporation, siRNA incorporation in hypothalamus stem cell exosomes can be obtained by direct loading of siRNA into the exosomes ex vivo, transfection, fusion of lipids and by transfection of cargo-loading chaperone proteins.

### Example 27: Blood Brain Barrier

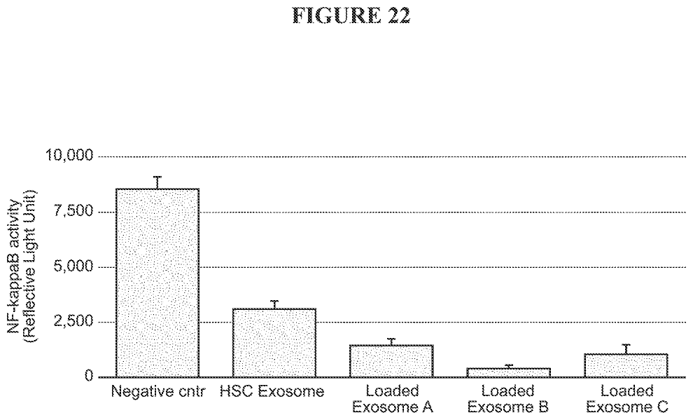

An in vitro model was used in FIG. 22 to show the ability of NFKB inhibitors to cross the blood-brain barrier following loading onto hypothalamus stem cell exosomes. HBEC-5i human cerebral endothelial cells were seeded at 50,000 cells/cm² on 0.1% gelatin coated, 24-well, 0.4 m-pore, translucent membrane inserts (0.3 cm²/insert) to establish a polarized monolayer representative of the BBB. Cells were cultured in DMEM:Fl2 medium supplemented with 40 μg/ml endothelial growth factor, 10% exosome-depleted fetal bovine serum, and I % penicillin/streptomycin solution. Human astrocytes were grown in DMEM:Fl2 Medium with 20 ng/ml human recombinant EGF, 20 ng/ml human recombinant basic FGF, and B-27 Supplement to a final concentration of 2% (v/v). CHLA-03-AA cells were seeded at 50,000 cells/cm² into the wells of the 24-well carrier plates (1.9 cm2/well). Cells were cultured in a humidified 37° C. incubator with 5% CO² until endothelial cells reached confluence. At confluence, endothelial cell inserts were co-cultured with CHLA-03-AA in DMEM:Fl2 Medium with 20 ng/ml human recombinant EGF, 20 ng/ml human recombinant basic FGF, and B-27 Supplement to a final concentration of 2%. 10 ng/ml recombinant human IL-I and 10 ng/ml recombinant human TNF-α was added to the culture along with mock-exosomes isolated from a no-cell negative control, or 50 ug of the following: human hypothalamus stem cell exosomes, human hypothalamus stem cell exosomes loaded with NFKB inhibitor 11-CAS 749886-87-1 (loaded exosome A), human hypothalamus stem cell exosomes loaded with NFKB inhibitor III-CAS 380623-76-7 (loaded exosome B), human hypothalamus stem cell exosomes loaded with NFKB inhibitor IV-CAS 139141-12-1 (loaded exosome C) for 24 hours. Hypothalamus stem cell exosomes were loaded with various NFKB inhibitors by incubation of exosomes with 200 uM of the respective inhibitor in a I:I v/v ratio for I hour at room temperature. After loading, the exosomes were separated from unincorporated drug using an exosome spin column (MW 3000). NFKB activation was assessed 24 hours after incubation by measuring activated nuclear NFKB protein.

Transfer of proteins across the blood brain barrier can be achieved by loading hypothalamus stem cell exosomes with proteins by transient or stable transfection, which is ideal for cytoplasmic proteins, or by using a vector that creates a chimeric protein that contains an exosome-anchoring protein such as HPV-E7 or HIV-I Nef.

Hypothalamus stem cell exosomes can also be loaded ex vivo with proteins by sonication, freeze-thaw cycles, treatment with saponin or extrusion. For saponin treatment, exosomes are diluted to 0.15 mg/ml, the protein is diluted in PBS (0.5 mg/ml) abd added to 250 μl of exosomes to a final concentration of 0.1 mg/ml total protein. The protein mixed with exosomes is supplemented with 0.2% saponin and placed on an orbital shaker for 20 min at RT. For freeze-thaw cycles, the protein solution is added to exosomes as described above, incubated for 30 min, then rapidly freezed at −80° C., and thawed at RT. The freeze-thaw cycle is repeated three times. For sonication, the protein is mixed with exosomes and sonicated (500 v, 2 kHz, 20% power, 6 cycles by 4 sec pulse/2 sec pause), cooled down on ice for 2 min, and then sonicated again. For extrusion, protein mixed with exosomes is extruded (×10 times) through an extruder with 200 nm-pore diameter. Exosomes loaded with protein are purified from free protein by gel-filtration chromatography or by column. While these methods are useful for proteins, they may also be used for nucleic acids, hormones and small molecule drugs.

It should be recognized that illustrated embodiments are only examples of the disclosed product and methods and should not be considered a limitation on the scope of the invention. Rather, the scope of the invention is defined by the following claims. We therefore claim as our invention all that comes within the scope and spirit of these claims.

1. A method for identifying, isolating and purifying human hypothalamus stem cells at an intermediate transitory stage of in vitro induced pluripotent stem cell differentiation, which corresponds to human hypothalamus stem cell formation, wherein the method comprises: (i) culturing induced pluripotent stem cell in culture media and enabling reproducible differentiation of induced pluripotent stem cells into cells of ventral diencephalon hypothalamic cell lineage; (ii) identifying hypothalamic cells between day O and day 15 of cell culture that display hypothalamus markers and neuronal stem cell markers; (iii) isolating hypothalamus stem cells at hypothalamus marker and neuronal stem cell marker maximal expression; and (iv) purifying isolated human hypothalamus stem cells.

2. The method of claim 1, wherein the neuronal stem cell markers are one or more of Sox2+, Bmi-1+, nestin+, Musashi and Cxcr4+_

3. The method of claim 2, wherein the hypothalamus markers comprise one or more of NK2 homeobox 1 (Nkx2.1) and homeobox protein orthopedia.

4. The method of claim 3, wherein the purified human hypothalamus stem cells are Raxhi, Soxl¹⁰, Sox2+ and Bmi-1+.

5. The method of claim 4, wherein hypothalamus stem cells at hypothalamus marker and neuronal stem cell marker maximal expression is between day 7 and day 15 of cell culture.

6. The method of claim 5, wherein the method further comprises analyzing the purified human hypothalamus stem cells for immunophenotype, neurosphere formation and ability to give rise to mature hypothalamus neurons.

7. The method of claim 6, wherein the ability to give rise to mature hypothalamus neurons is measured by detecting expression of one or more neuropeptide markers.

8. The method of claim 7, wherein the one or more neuropeptide markers comprise Otp, Rax, neuropeptide Y (NPY), cocaine amphetamine regulated transcript (CART), a-melanocyte stimulating hormone (a-MSH), neuropeptide Y receptor Y2 (NPYR), ghrelin receptor (GhrR), and melanin concentrating hormone (MCH).

9. The method of claim 1, wherein the method further comprises isolating and purifying exosomes from the culture media, and analyzing purified exosomes by one or more of western blot analysis, flow cytometry, nanoparticle-tracking analysis, density-gradient ultracentrifugation, pull-down assay, and real-time PCR.

10. Purified human hypothalamus exosomes obtained by the method of claim 9.

11. The method of claim 9, wherein the method further comprises engineering purified exosomes to express one or more foreign molecules.

12. The method of claim 10, wherein the one or more foreign molecules comprise one or more of a growth factor, a nucleic acid, a cytokine, a vaccine, a inactivated viral protein, a drug, a chemotherapeutic and a biologically active molecule.

13. The method of claim 11, where the nucleic acid is DNA, messenger RNA, micro RNA, or small interfering RNA

14. The method of claim 12, wherein the method further comprises administering to a subject in need thereof a pharmaceutical composition comprising engineered exosomes for preventing, treating or controlling a disease or disorder associated with hypothalamus malfunction in the subject.

15. The method of claim 14, wherein the disease or disorder is one or more of an infection, a viral disease, a degenerative disease, an autoimmune disease, a genetic disorder, a dermatological disorder, a topical inflammation, a metabolic syndrome, a cancer, or a damaged tissue in need of repair.

16. A method for identifying a drug that modulates hypothalamus function in a subject in need thereof, wherein the method comprises (a) transfecting the purified human hypothalamus stem cells of claim 5 with a reporter gene operably linked to a transcriptional regulatory element isolated from the subject and regulating expression of the reporter gene; (b) contacting transfected human hypothalamus stem cells with a candidate drug; (c) determining drug effect on transcriptional regulatory element expression by detecting and measuring a signal emanating from the reporter gene and comparing the signal to a signal emanated by the reporter gene prior to contact with the drug, thereby identifying a drug that modulates hypothalamus function in the subject.

17. The method of claim 16, wherein drug effect is further determined by measuring one or more of cell viability, proliferation, apoptosis, stem-ness, and differentiation.

18. The method of claim 17, wherein the drug decreases neuroinflammation, alters circadian rhythm, regulates thyroid function or affects obesity, inflammation, infertility and aging.

19. The method of claim 18, wherein the subject is a mammal.

20. The method of claim 19, wherein the subject is a human subject.


SEQUENCE LISTING (16 sequences)

SEQ ID NO: 1 - DNA, 22 bp, artificial sequence
  synthetic construct
ctcttctgcctgctgcactttg

SEQ ID NO: 2 - DNA, 22 bp, artificial sequence
  synthetic construct
atgggctacaggcttgtcactc

SEQ ID NO: 3 - DNA, 21 bp, artificial sequence
  synthetic construct
gacagccactcacctcttcag

SEQ ID NO: 4 - DNA, 22 bp, artificial sequence
  synthetic construct
ttctgccagtgcctctttgctg

SEQ ID NO: 5 - DNA, 22 bp, artificial sequence
  synthetic construct
ccacagaccttccaggagaatg

SEQ ID NO: 6 - DNA, 23 bp, artificial sequence
  synthetic construct
gtgcagttcagtgatcgtacagg

SEQ ID NO: 7 - DNA, 22 bp, artificial sequence
  synthetic construct
gcagcactacttcttgaccacc

SEQ ID NO: 8 - DNA, 22 bp, artificial sequence
  synthetic construct
tctgctcctgagcattgacgtc

SEQ ID NO: 9 - DNA, 23 bp, artificial sequence
  synthetic construct
ctaagcggaatgagcagaggatc

SEQ ID NO: 10 - DNA, 22 bp, artificial sequence
  synthetic construct
tctcaacagccactcacgcaca

SEQ ID NO: 11 - DNA, 22 bp, artificial sequence
  synthetic construct
caccattggcaatgagcggttc

SEQ ID NO: 12 - DNA, 22 bp, artificial sequence
  synthetic construct
aggtctttgcggatgtccacgt

SEQ ID NO: 13 - DNA, 20 bp, artificial sequence
  synthetic construct
actccgtgaaggcaaagaga

SEQ ID NO: 14 - DNA, 20 bp, artificial sequence
  synthetic construct
caagatccggagatgttggt

SEQ ID NO: 15 - DNA, 21 bp, artificial sequence
  synthetic construct
ggcaaatgctggacccaacac

SEQ ID NO: 16 - DNA, 21 bp, artificial sequence
  synthetic construct
tgccattcctggacccaaagc